(19) United States
(12) Patent Application Publication
(10) Pub. No.: US 2023/0309868 A1
(43) Pub. Date: Oct. 5, 2023

(54) PHOTO-ACTIVATED FLUORESCENCE SENSOR

(71) Applicant: Fariborz TAGHIPOUR, Burnaby (CA)

(72) Inventor: Fariborz TAGHIPOUR, Burnaby (CA)

(21) Appl. No.: 18/130,863

(22) Filed: Apr. 4, 2023

Related U.S. Application Data
(60) Provisional application No. 63/327,037, filed on Apr. 4, 2022.

(51) Int. Cl. A61B 5/1455; G01N 21/64; B01L 3/00

(52) U.S. Cl. CPC A61B 5/1455; G01N 21/6428; B01L 3/502715; G01N 2021/6439; B01L 2300/0636; B01L 2200/0647; A61B 5/14521

(57) ABSTRACT

A sensor for detection or quantitative measurement of a first target molecule in an analyte comprises: a first sensing node provided in solid phase on a solid-phase substrate, the first sensing node comprising a first radiation-activatable fluorescence material and a first recognition element for interaction with the first target molecule; and a radiation emitter optically configured to direct input radiation toward the first sensing node. The first radiation-activatable fluorescence material is fluoresce-able in response to interaction with the input radiation to thereby cause the first sensing node to emit first output radiation. One or more spectral characteristics of the first output radiation are detectably influence-able in response to interaction between the first recognition element and the first target molecule.

### REFERENCE TO RELATED APPLICATIONS

This application claims priority from, and for the purposes of the United States the benefit of 35 USC § 119 in respect of, U.S. application No. 63/327,037 filed 4 Apr. 2022, which is hereby incorporated herein by reference.

### TECHNICAL FIELD

This invention related to sensors incorporating radiation-activatable fluorescence materials. Particular embodiments provide sensors incorporating radiation-activatable fluorescence materials for detection or quantitative measurement of a first target molecule in an analyte.

### BACKGROUND

In general terms, a sensor is a device, module, machine, or subsystem whose purpose is to detect events or changes in its environment. A chemical sensor may be considered to be a device that transforms chemical information (composition, presence of a particular element or ion, concentration, chemical activity, etc.) into a signal.

One type of chemical sensor is a biosensor. A biosensor may be considered to be an analytical device comprising a biological sensing element. A biosensor may harness the sensitivity and specificity of biology in conjunction with physicochemical detectors to deliver bioanalytical measurements or signals. Biosensors could provide critical insights into the performance and health of living organisms (e.g., humans, other animals, plants or other living organisms).

Chemical sensors may comprise: a recognition element (also referred to as a receptor) that interacts with (or binds with, or otherwise recognizes) the target molecule in an analyte under study; and a detection element (also referred to as transducer) that converts this interaction into a measurable signal. The signal output from a chemical sensor can be measured, amplified, otherwise processed, displayed by a suitable display device, interpreted and/or the like. Existing sensors working based on such principles have several challenges. There is a general need for improved chemical sensors and/or biosensors.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a sensor for detection or quantitative measurement of a first target molecule in an analyte. The sensor comprises: a first sensing node provided in solid phase on a solid-phase substrate, the first sensing node comprising a first radiation-activatable fluorescence material and a first recognition element for interaction with the first target molecule; a radiation emitter optically configured to direct input radiation toward the first sensing node. The first radiation-activatable fluorescence material is fluoresce-able in response to interaction with the input radiation to thereby cause the first sensing node to emit first output radiation. One or more spectral characteristics of the first output radiation are detectably influence-able in response to interaction between the first recognition element and the first target molecule.

The first recognition element may comprise one or more first recognition sites with an affinity for the target molecule.

The sensor may comprise a housing. The radiation emitter and the first sensing node may be located at least partially within the housing. The first sensing node may be attached to a wall of the housing.

The one or more spectral characteristics of the first output radiation may comprise radiation intensity at one or more wavelengths.

The sensor may comprise a detector. The detector may be optically configured to capture the one or more spectral characteristics of the first output radiation. The detector may comprise at least one of: a radiation detector, a light detector, a color detector, an image detector, a digital image sensor, a CCD sensor and a CMOS sensor.

The sensor may comprise at least one of: a narrowband filter, a broadband filter, a bandpass filter, a bandstop filter, a UV pass filter, a UV cut filter, a visible light pass filter, a visible light cut filter and a QDs filter located in at least one of: a first location between the detector and the first sensing node, a second location between the radiation emitter and the detector and a third location between the radiation emitter and the first sensing node.

The sensor may comprise a second sensing node provided in solid phase on the solid-phase substrate. The second sensing node may comprise a second radiation-activatable fluorescence material and a second recognition element for interaction with a second target molecule. The radiation emitter may be optically configured to direct the input radiation toward the second sensing node. The second radiation-activatable fluorescence material may be fluoresce-able in response to interaction with the input radiation to thereby cause the second sensing node to emit second output radiation. One or more spectral characteristics of the second output radiation may be detectably influence-able in response to interaction between the second recognition element and the second target molecule.

The first recognition element may comprises a first type of recognition site with an affinity for the first target molecule and the second recognition element may comprise a second type of recognition site with an affinity for the second target molecule. The first recognition element may be different in shape or chemical composition from the second recognition element. The first recognition element may comprise a first type of recognition site with an affinity for the first target molecule and the second recognition element may comprise a second type of recognition site with an affinity for the second target molecule. The first target molecule my be different in chemical composition from the second target molecule.

The sensor may comprise a second sensing node provided in solid phase on the solid-phase substrate. The second sensing node may comprise a second radiation-activatable fluorescence material. The radiation emitter may be optically configured to direct the input radiation toward the second sensing node. The second radiation-activatable fluorescence material may be fluoresce-able in response to interaction with the input radiation to thereby cause the second sensing node to emit second output radiation. One or more spectral characteristics of the second output radiation are detectable in response to interaction between the second sensing node and the analyte.

The first sensing node may be provided on a first side of the solid-phase substrate. The solid-phase substrate may be at least partially transparent to the input radiation emitted by the radiation emitter. The radiation emitter may be configured to direct the input radiation toward the first sensing node through the substrate from a second side of the substrate, the second side of the solid-phase substrate different from (e.g. opposite to) the first side of the solid-phase substrate.

The solid-phase substrate may be provided on a substrate side of the first sensing node. The radiation emitter may be configured to direct the input radiation toward a target side of the sensing node, the target side of the sensing node different from (e.g. opposite to) the substrate side of the sensing node.

At least a portion of the housing may be at least partially transparent to the first output radiation such that the one or more spectral characteristics of the first output radiation are detectable through the at least a portion of the housing that is at least partially transparent. The sensor may comprise a detector optically configured to detect the one or more spectral characteristics of the first output radiation through the at least a portion of the housing that is at least partially transparent and wherein the detector comprises at least one of a radiation detector, a light detector, a color detector, an image detector, a digital image sensor, a CCD sensor and a CMOS sensor. The sensor may comprise a detector optically configured to detect the one or more spectral characteristics of the first output radiation through the at least a portion of the housing that is at least partially transparent and wherein the detector comprises a digital image sensor of a mobile computing device. The mobile computing device may comprise a digital camera, a tablet, a camera phone, a smartphone, a tablet computing device, a smart watch or a smart wearable device.

A vector of a principal emission direction of the input radiation may be substantially parallel with a vector normal to a plane defined by a broad surface of the first sensing node.

A vector of a principal emission direction of the input radiation may be non-parallel with a vector normal to a plane defined by a broad surface of the first sensing node.

A vector of a principal emission direction of the input radiation may intersect with a vector normal to a plane defined by a broad surface of the first sensing node by an angle of less than 40 degrees.

The radiation emitter may be located to irradiate the first sensing node from a substrate side of the sensing node. A detector may be located on the substrate side of the sensing node to receive first output radiation from the substrate side of the sensing node. The sensing node may be located to interact with the analyte on a target side of the sensing node, the target side of the sensing node opposite the substrate side of the sensing node.

The radiation emitter may be located on to irradiate the first sensing node from a target side of the sensing node. A detector may be located to receive first output radiation from the target side of the sensing node. The sensing node may be located to interact with the analyte on the target side of the sensing node.

The radiation emitter may be located to irradiate the first sensing node from a substrate side of the sensing node. A detector may be located to receive first output radiation from a target side of the sensing node. The sensing node may be located to interact with the analyte on the target side of the sensing node, the target side of the sensing node opposite the substrate side of the sensing node.

The sensor may comprise an optical lens positioned in an optical path of the first output radiation between the first sensing node and a detector. The detector may be configured to measure the one or more spectral characteristics of the first output radiation.

The sensor may comprise a battery to power the radiation emitter and the detector.

The sensor may comprise a repellant module for repelling a first target molecule bound to the first sensing node from the first sensing node. The repellant module may comprise at least one of: a pair of electrodes, laser-engraved graphene (LEG), and redox-active nanoreporters (RARs).

The sensor may comprise a release module for stimulating the release of biofluids from skin. The release module may comprise an iontophoresis module.

The first sensing node provided on the solid-phase substrate may be removable and replaceable with a second sensing node provided in a solid phase on a second solid-phase substrate, the second sensing node comprising a second radiation-activatable fluorescence material and a second recognition element for interaction with a second target molecule. The radiation emitter may be optically configured to direct the input radiation toward the second sensing node. The second radiation-activatable fluorescence material may be fluoresce-able in response to interaction with the input radiation to thereby cause the second sensing node to emit second output radiation. One or more spectral characteristics of the second output radiation may be detectably influence-able in response to interaction between the second recognition element and the second target molecule.

The radiation emitter may comprise a solid-state UV emitter. The solid-state UV emitter may comprise an ultraviolet light emitting diode (UV-LED).

The first radiation-activatable fluorescence material may comprise one or more types of quantum dots. The one or more types of quantum dots may comprise at least two types quantum dots. The at least two types of quantum dots may comprise types of quantum dots of different chemical compositions, size or shape. The one or more one types of quantum dots may comprise at least one type of quantum dot having a chemical composition selected from the group consisting of: zinc oxide (ZnO), cadmium sulfide (CdS), cadmium selenide (CdSe), zinc selenide (ZnSe), indium phosphide (InP), carbon, and graphene.

The recognition element may comprise an imprinted polymer (IP). The imprinted polymer may comprise a molecularly imprinted polymer (MIP) or a surface imprinted polymer (SIP). The imprinted polymer may comprise at least one of: 3-Aminopropyltriethoxysilane (APTES) or 5-indolyl boronic acid.

The first radiation-activatable fluorescence material may be doped with at least one of: metal particles, non-metal particles, a catalyst and a polymer.

The sensor may comprise at least one of a porous material, microporous material, mesoporous material, macroporous material, ordered hierarchical porous material, structure-directing surfactant, sulfonated tetrafluoroethylene based fluoropolymer-copolymer, crosslinker agent, graphene derivatives, active fluorescent quencher, absorbent path, and membrane integrated with the first sensing node or located between the first sensing node and an analyte-receiving surface of the sensor.

The radiation emitter may be configurable to emit radiation of at least one of: a plurality of different intensities and a plurality of different wavelengths.

The radiation emitter may be configurable to emit radiation at different intensities.

The radiation emitter my comprise a plurality of radiation sub-emitters, wherein at least two sub-emitters are configurable to emit radiation at different wavelengths.

The sensor may comprise: a second sensing node provided in solid phase on the solid-phase substrate, the second sensing node comprising a second radiation-activatable fluorescence material and a second recognition element for interaction with a second target molecule; and a second radiation emitter optically configured to direct second input radiation toward the second sensing node. The second radiation-activatable fluorescence material may be fluoresce-able in response to interaction with the second input radiation to thereby cause the second sensing node to emit second output radiation. One or more spectral characteristics of the second output radiation may be detectably influence-able in response to interaction between the second recognition element and the second target molecule. The input radiation and the second input radiation may have different intensities and/or different wavelengths.

The sensor may be integrated into at least one of: a laptop, a mobile phone, a watch, and a wearable device.

The first sensing node may be fabricated on at least one of: a UV-LED chip, a UV-LED wafer and a UV-LED package.

The first sensing node is fabricated on at least one of: paper and polymer sheet substrate.

Another aspect of the invention provides use of any of the sensors described herein for detecting a presence of, or estimating a quantity of, a target molecule in the analyte.

The target molecule may be or comprises a biomarker, such as glucose, lactate, dopamine, and/or cortisol, and the analyte may be or comprise a biofluid, such as sweat, blood, saliva, mucus, urine, stool and/or interstitial fluid. The first target molecule may be or comprise a pollutant, such as toxic compounds, chemical hazards, or environmental contaminants, and the analyte may be or comprise air or water.

Another aspect of the invention provides a method for detecting a presence or quantity of a target molecule in an analyte using a sensor. The method comprises: establishing contact between the analyte and the first sensing node of any of the sensors described herein; and detecting one or more of the one or more spectral characteristics of the first output radiation.

The one or more of the one or more spectral characteristics of the first output radiation may comprise at least one of: light intensity, light spectrum, light brightness, and a value corresponding to a relative color intensity of at least one of the colors of red, green, blue, cyan, magenta, yellow, and key.

The method may comprise detecting a presence or quantity of the first target molecule in the analyte by employing an artificial intelligence engine trained by machine learning or deep learning to detect the presence or quantity of the target molecule in the analyte based at least in part on the one or more spectral characteristics of the first output radiation.

Another aspect of the invention provides a device for detection or quantitative measurement of a first target molecule in an analyte using the camera of a mobile computing device. The device comprises: a radiation emitter supported in a housing and controllable to emit input radiation; and a solid-phase substrate comprising a first sensing node provided in solid phase on the substrate for exposure to the analyte, the first sensing node comprising a first radiation-activatable fluorescence material and a first recognition element for interaction with the first target molecule in the analyte. The solid-phase substrate is insertable into the housing in a location where the input radiation impinges on the first sensing node. The first radiation-activatable fluorescence material is fluoresce-able in response to interaction with the input radiation to thereby cause the first sensing node to emit output radiation. One or more spectral characteristics of the output radiation are detectably influence-able in response to interaction between the first recognition element and the first target molecule. The device is mountable, or otherwise locatable, relative to the camera of the mobile computing device such that at least some of the output radiation exits the housing through an aperture and is detectable by the camera.

The mobile computing device may comprise a digital camera, a tablet, a camera phone, a smartphone, a tablet computing device, a smart watch or a smart wearable device.

The one or more spectral characteristics may comprise radiation intensity of the output radiation at one or more wavelengths.

The device may comprise any of the features, combinations of features and/or sub-combinations of features of any of the other devices or sensors described herein.

Another aspect of the invention provides a wearable device for detection or quantitative measurement of a first target molecule in an analyte. The wearable device comprisies: an image sensor supported in a wearable housing; and a substrate comprising a first sensing node provided on the substrate, the first sensing node comprising a first radiation-activatable fluorescence material and a first recognition element for interaction with the first target molecule in the analyte. The substrate is mountable to an exterior of the wearable housing for exposure to the analyte. A radiation emitter is supported in the wearable housing and controllable to emit input radiation onto the first sensing node. The first radiation-activatable fluorescence material is fluoresce-able in response to interaction with the input radiation to thereby cause the sensing node to emit output radiation. One or more spectral characteristics of the output radiation are detectably influence-able in response to interaction between the first recognition element and the first target molecule. At least some of the output radiation is detectable by the image sensor.

The substrate may be mountable to an exterior of the housing for exposure to the analyte when the device is being worn.

The image sensor may comprise a CMOS image sensor or a CCD image sensor.

The one or more spectral characteristics may comprise radiation intensity of the output radiation at one or more wavelengths.

The device may comprise an optical system for at least one of: directing the input radiation onto the first sensing node and directing the output radiation toward the image sensor. The optical system may comprise one or more optical elements selected from the group consisting of: mirrors, lenses, fiber optics, prisms, transparent windows and transparent walls.

The device may comprise any of the features, combinations of features and/or sub-combinations of features of any of the other devices or sensors described herein.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1A is a schematic depiction of a sensing device, according to an example embodiment of the invention. FIG. 1B is a schematic depiction of use of a sensing device, according to an example embodiment of the invention.

FIG. 2 depicts an isometric view of sensing device, according to an example embodiment of the invention.

FIG. 3A depicts a side view of the sensing device of FIG. 2. FIG. 3B depicts a cross-section of the device of FIG. 3A taken along A-A.

FIG. 4A is a schematic depiction of a cross-section of a portion of a sensing device mounted to a mobile computing device according to an example embodiment of the invention. FIG. 4B is a schematic depiction of the mobile computing device of FIG. 4A.

FIG. 5 is a perspective view of a wearable sensing device, according to an example embodiment of the invention.

FIG. 6A is a schematic depiction of a cross-section of a portion of the wearable sensing device of FIG. 5. FIG. 6B is a schematic depiction of a top view of a portion of the wearable sensing device of FIG. 5.

FIG. 7 is a schematic depiction of another sensing device, according to an example embodiment of the invention.

FIG. 8 is a schematic depiction of another sensing device, according to an example embodiment of the invention.

FIG. 9 is a schematic depiction of another sensing device, according to an example embodiment of the invention.

FIG. 10 is a schematic depiction of another sensing device, according to an example embodiment of the invention.

FIG. 11 is a schematic depiction of another sensing device, according to an example embodiment of the invention.

FIG. 12 is a schematic depiction of another a sensing device, according to an example embodiment of the invention.

FIG. 13 is a schematic depiction of another sensing device, according to an example embodiment of the invention.

FIG. 14 is a schematic depiction of the use of a sensing device, according to an example embodiment of the invention.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

One aspect of the invention provides a sensor for detection or quantitative measurement of a first target molecule in an analyte. The sensor may comprise a first sensing node provided in solid phase on a solid-phase substrate and a radiation emitter optically configured to direct input radiation toward the first sensing node. The first sensing node may comprise a first radiation-activatable fluorescence material and a first recognition element for interaction with the first target molecule. The first recognition element may comprise one or more first recognition sites to encourage the first target molecule to interact with the first recognition element. The first radiation-activatable fluorescence material may be fluoresce-able in response to interaction with the input radiation to thereby cause the first sensing node to emit first output radiation. One or more spectral characteristics of the first output radiation may be detectably influence-able in response to interaction between the first recognition element and the first target molecule.

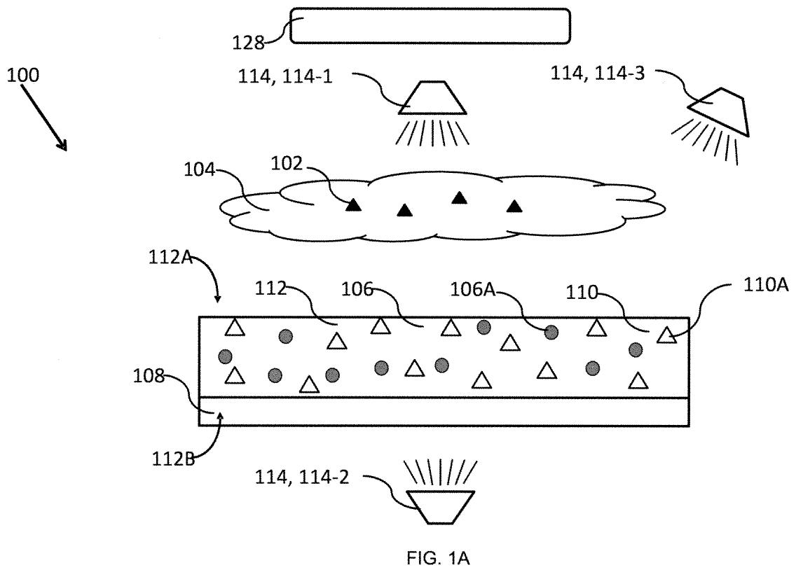

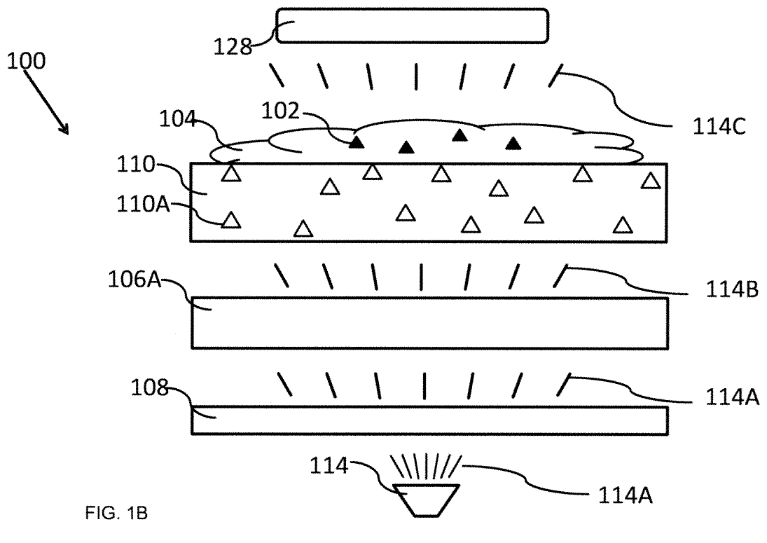

FIG. 1A is a schematic diagram of an exemplary sensor 100 for detection or quantitative measurement of one or more target molecules 102 in an analyte 104. Sensor 100 may be employable to detect the mere presence of target molecule(s) 102 in analyte 104 (e.g. at any quantity, or above a threshold quantity) or to quantitatively measure a quantity or concentration of target molecules(s) 102 in analyte 104.

Analyte 104 may comprise, a liquid, a gas or a solid. Analyte 104 may comprise, for example, a biofluid such as, but not limited to, sweat, blood, saliva, mucus, urine, stool, interstitial fluid (e.g. the body fluid between blood vessels and cells), etc. Analyte 104 may comprise water, soil or air.

Target molecule(s) 102 may comprise any suitable target molecules. For example, target molecule(s) 102 may comprise biomarkers, pollutants, nutrients, etc. The composition of target molecule(s) 102 may be dependent on the composition of analyte 104. For example, where analyte 104 comprises a biofluid, target molecule 102 may comprise a biomarker such as, not limited to, metabolites, nutrients, glucose, lactate, dopamine, and/or cortisol. In this way, sensor 100 may provide for dynamic, non-invasive measurements of biochemical markers in biofluids thereby allowing the monitoring of physiological health status, disease diagnostics and health management. As another example, where analyte 104 comprises water or air, target molecule 102 may comprise one or more pollutants (e.g. toxic compounds, chemical hazards, environmental contaminants, etc.). As a further example, where analyte 104 comprises soil, target molecule 102 may comprise nutrients in soil, such as nitrogen and/or nitrates in soil.

Sensor 100 may comprise a sensing layer 112 comprising one or more sensing nodes 106 provided on a substrate 108. Sensing layer 112 and/or sensing nodes 106 may be provided in a solid phase. Solid-phase sensing layer 112 and/or solid-phase sensing nodes 106 may exhibit a relatively high sensitivity (even at low target molecule concentrations within an analyte) as compared to laboratory-based liquid-phase sensing materials. This relatively high sensitivity may be because solid-phase sensing layer 112 and/or solid-phase sensing nodes 106 may mitigate or prevent dilution of the analyte. Solid-phase substrate 108 may provide a support structure for solid-phase sensing nodes 106 which may in turn provide advantages of each of immobilizing solid-phase sensing node(s) 106 and replacement of sensing nodes 106 after use. Further, solid-phase sensing layer 112 and/or solid phase sensing nodes 106 may be advantages for ease of use and transportation/storage.

Each sensing node 106 may comprise a radiation-activatable fluorescence material 106A (also referred to herein as a sensing material 106A). Sensing material 106A radiation-may be fluoresceable in response to interaction with input radiation of one or more radiation sources 114 (discussed further herein). Radiation-activatable fluorescence material 106A may be in the form of one or more quantum dots. Quantum dots may comprise very small semiconductor particles (e.g. of a few nanometres in diameter). When the quantum dots are irradiated by radiation (e.g. visible light or UV radiation), an electron in the quantum dot may be excited to a state of higher energy, which leads to emitting light. One or more spectral characteristics of the light (e.g. colour, radiation intensity at one or more wavelengths, etc.) emitted from the quantum dot may be dependent the shape and/or size of the quantum dots due to quantum confinement. Quantum dots may comprise, for example, zinc oxide (ZnO), cadmium sulfide (CdS), cadmium selenide (CdSe), zinc selenide (ZnSe), indium phosphide (InP), carbon quantum dots, graphene quantum dots, or a combination thereof.

The quantum dots may have different structurers and morphologies. For example, the quantum dots may comprise core-shell quantum dots. Different structure and morphologies may enhance sensitivity or response time of the quantum dots by changing surface area and/or electron conductivity of the sensing material. In some embodiments, the quantum dots may be combined, integrated, or capped (e.g. core-shell structure) by other material to enhance their functionality and/or lifetime.

In some embodiments, a dye such as organic dye or quantum dots of different colors may be integrated with the sensing material 106A. Using non-target-specific emissive quantum dots and dyes may lead to generating more distinctive colour images and enhance the colour and intensity detection by the detector and analysis software.

Each sensing node 106 may also comprise a recognition element 110. Recognition element 110 may comprise a material configured to interact with target molecule(s) 102 such that one or more spectral characteristics of output radiation 114C of sensing material 106A may be detectably influence-able in response to interaction between the first recognition element and the first target molecule. In other words, interaction between target molecule(s) 102 and recognition element 110 may cause radiation outputted by sensing node 106 to have one or more different spectral characteristics as compared to if target molecule(s) 102 are not present and interacting with recognition element 110.

Recognition element 110 may comprise one or more recognition sites 110A (also referred to herein as imprinted sites 110A or sites 110A) configured to encourage target molecule(s) 102 to interact with (e.g. bind to, adhere to or otherwise recognize) recognition element 110. Sites 110A may comprise one or more elements configured to discourage other molecules (e.g. molecules other than target molecule(s) 102) to bind to, adhere to or otherwise interact with recognition element 110. Sites 110A may be chosen based at least in part on the type of target molecule(s) 102. Where multiple different types of target molecules 102 are targeted, different sites 110A may be provided for each type of target molecule 102. Fluorescence sensing material 106A (e.g. quantum dots), may be combined with (e.g. at least partially covered in, impregnated with, in contact with, and/or otherwise interact with) recognition element 110 including sites 110A.

Recognition element 110 may comprise, for example, biological recognition elements, such as receptors, biomolecules, imprinted polymers, nucleic acids, whole cells, antibodies, different classes of enzymes and/or the like. Combining sensing material 106A with such biological recognition elements may facilitate the reaction and/or interaction with particular target molecules 102. Combining sensing material 106A with sites 110A of recognition element 110 may additionally or alternatively enhance the selectivity of sensing material 106A for detection of one or more specific materials, molecules, biomarkers and/or the like in analyte 104.

Recognition element 110 may comprise imprinted polymer, such as molecularly imprinted polymer (MIP) or surface imprinted polymer (SIP), having template-induced cavities as sites 110A. Imprinted polymers may be synthetic polymers formed with the existence of the target molecule(s) 102 and appropriate monomers. The imprinted sites 110A formed during the polymerization may match target molecule(s) 102 in terms of shape, size, and/or functional group. Therefore, these imprinted sites 110A may selectively or preferably bind to target molecule(s) 102 when re-exposed to the molecule used in fabrication. The imprinted polymers may also be formed using dummy molecules (molecules other than the actual target molecules 102) or using a functional group of target molecule(s) 102. Examples of imprinted polymers include polymers formed from 3-Aminopropyltriethoxysilane monomer or 5-indolyl boronic acid monomer. Initiators may be used in the process of polymerization of recognition element 110 to enhance the process. The imprinted polymers relate to target molecule(s) 102 or components thereof by different means such as its functional groups, adsorption affinity, shape and/or size. Due to sites 110A of recognition element 110, when sensor 100 is exposed to analyte 104, mainly target molecule(s) 102 are able to bind to recognition element 110.

At least a portion of sensing layer 112 (e.g. sensing material 106A and/or recognition element 110) may be decorated/doped with metal particles (e.g. nano-particles), such as platinum, gold, silver and/or the like, and/or one or more compositions of metal particles and/or metal oxide particles, such as manganese dioxide (MnO₂) and/or the like. Sensing layer 112 (e.g. sensing material 106A and/or recognition element 110) may be decorated/doped with non-metal particles (e.g. nano-particles) and/or combinations of non-metal particles, such as graphitic carbon nitride (g-C₃N₄), fluorine (F) and/or the like. Sensing layer 112 (e.g. sensing material 106A and/or recognition element 110) may be decorated/doped with one or more catalysts, such as dissociation, oxidation, adsorption catalysts and/or the like. Sensing material 106A (e.g. quantum dots) may be functionalized (or surface functionalized) with one or more active chemicals, organometallic compounds and/or the like. Decorating/doping of sensing layer 112 (e.g. sensing material 106A and/or recognition element 110) with metal particles, non-metal particles, catalysts, functional groups and/or the like, may enhance sensitivity, selectivity, or response time of sensing nodes 106 by changing the reaction sites, and/or electrical and optical characteristics of sensing layer 112 (e.g. sensing material 106A and/or recognition element 110).

At least a portion of sensing layer 112 (e.g. sensing material 106A and/or recognition element 110) may be combined with one or more electron conductive materials, such as graphene, and/or graphene derivatives, such as graphene oxide, reduced graphene oxides and/or the like. Combining sensing layer 112 (e.g. sensing material 106A and/or recognition element 110) with electron conductive material such as graphene and/or graphene derivatives may enhance sensitivity or response time of sensing nodes 106 by changing the electron conductivity and/or other electrical characteristics of sensing layer 112 (e.g. sensing material 106A and/or recognition element 110).

At least a portion of sensing layer 112 (e.g. sensing material 106A and/or recognition element 110) may be combined with receptors, nucleic acids, whole cells, antibodies and different classes of enzymes. Combining sensing layer 112 (e.g. sensing material 106A and/or recognition element 110) with biomolecules, cells, and enzymes may facilitate the reaction with the biomarkers (for biosensor applications).

At least a portion of sensing layer 112 (e.g. sensing material 106A and/or recognition element 110) may be made with a solution that has similar properties (physical and/or chemical characteristics, for example salinity, pH, and the like) to those of target molecule 102 and/or analyte 104 to adapt sensing layer 112 (e.g. sensing material 106A and/or recognition element 110) for the analyte environment. For example, in the process of synthesizing a sensing layer 112 (e.g. sensing material 106A and/or recognition element 110) for measuring lactate in sweat, phosphate-buffered saline (PBS), which is a water-based, a salt solution may be used (e.g. 50% of PBS solution may be used to prepare the sensing layer) to mimic the ion-rich saline content of sweat, and therefore to adapt the sensing material 106A for using in the sweat saline environment.

In some embodiments, at least a portion of sensing layer 112 (e.g. sensing material 106A and/or recognition element 110) is combined, integrated, or covered with porous material, including microporous, mesoporous, macroporous materials, and/or other general and/or ordered hierarchical porous materials. For example, in some embodiments, at least a portion of target side 112A of sensing layer 112 is combined, integrated, or covered with porous material, including microporous, mesoporous, macroporous materials, and/or other general and/or ordered hierarchical porous materials. This structure may improve the absorbance of material on sensing layer 112 (e.g. sensing material 106A and/or recognition element 110) by providing a higher surface area or volume with which target molecules 102 may bind or interact. Additionally or alternatively, the microporous, mesoporous, macroporous and/or ordered hierarchical materials may act as a filter to prevent the diffusion of one or more undesired molecules to sensing layer 112 (e.g. sensing material 106A and/or recognition element 110). These porous materials may control the diffusion rate of one or more undesired molecules to sensing layer 112. The prevention and/or mitigation of diffusion of one or more undesired molecules may enhance the selectivity, sensitivity, detection capability and/or response time of sensing layer 112 by blocking and/or delaying some interfering molecules from reaching sensing layer 112. Controlling the diffusion may additionally or alternatively enhance the selectivity, sensitivity, detection capability and/or response time of sensing layer 112 by separating particular molecules to interact at different times with sensing layer 112, thereby permitting identification and/or quantification of each molecule separately. The porous material may additionally or alternatively enhance the transfer of some target molecules 102 to sensing layer 112. In some embodiments, at least a portion of sensing layer 112 (e.g. at least a portion of target side 112A) may be combined or covered with chitosan or sulfonated tetrafluoroethylene-based fluoropolymer-copolymer (e.g. Nafion™) which may provide enhanced protection for sensing layer 112 and/or enhanced functionality for sensing layer 112.

In some embodiments, at least a portion of sensing layer 112 (e.g. sensing material 106A and/or recognition element 110) is combined, integrated, or covered with a structure-directing surfactant (e.g. cetyltrimethylammonium bromide (CTAB)). This combination may produce porous or mesoporous structure that extending the surface of the imprinted polymer, thus improving diffusion of target molecules 102.

In some embodiments, at least a portion of sensing layer 112 (e.g. sensing material 106A and/or recognition element 110) is combined, integrated, or cover a perforated or a porous membrane positioned on the surface of sensing layer 112 to block interfering macromolecules. The membrane may be a replicable/disposable membrane that could be changed.

At least a portion of sensing layer 112 (e.g. sensing material 106A and/or recognition element 110) may comprise other chemicals such as functional monomers and crosslinking agents (e.g. tetraethyl orthosilicate (TEOS)). The crosslinking agent may be applied to control the structure of the polymer matrix of the imprinted polymer of recognition element 110, in which such agents lead to aggregation and connection of functional monomers to each other and stand firmly in own place and polymerization and molecular template separation.

In some embodiments, sensing layer 112 (e.g. sensing material 106A and/or recognition element 110) may be in the form of a solid material provided on a substrate 108. In some embodiments, sensing layer 112 (e.g. sensing material 106A and/or recognition element 110) may be in the form of a paste or gel on substrate 108. In some embodiments, sensing layer 112 may be placed on or immobilized on substrate 108. This configuration may be advantageous for many reasons including ease of contacting sensing node(s) 106 with analyte 104, the ability to place several sensing nodes 106 on a single substrate 108 for measuring multiple different target molecules 102, ease of inserting sensing node(s) 106 inside a sensor device, and handling, storage, and transportation thereof.

The fabrication (e.g. placement and immobilization) of sensing layer 112 comprising one or more sensing nodes 106 on substrate 108 may be achieved by coating (e.g. physical coating such as, for example, spin coating) or chemical binding. In some embodiments, various sensing layers 112 and/or sensing nodes 106 (e.g. each having different sensing materials 106A and/or recognition elements 110 and/or combinations thereof) may be printed on a single substrate 108. The printing of sensing layer(s) 112 may be done through material printing (e.g. substantially similar to inkjet printing) where a liquid or paste, or solid powder phase of the material (e.g. sensing layer 112, sensing material 106A and/or recognition elements 110) are printed on substrate 108.

Substrate 108 may comprise any suitable material. In some embodiments, substrate 108 comprises, for example, a paper, a polymer sheet, or the like. In some embodiments, substrate 108 comprises a radiation transparent material (e.g. a material that allows radiation to at least partially pass through it). For example, substrate 108 may comprise a UV transparent material, such as transparent paper, polymer sheet, glass, quartz and/or the like.

In some embodiments, substrate 108 may be an absorbent path (e.g. a liquid permeable or penetrable path, such as water permeable path). In some embodiment, an absorbent path (e.g. UV transparent and/or visible light transparent path) may be applied on target side 112A of sensing layer 112 (e.g. opposite substrate side 112B where substrate 108 contacts sensing layer 112). The utilization of absorbent path may facilitate controlling providing a desired amount of analyte 104 into contact with sensing layer 112. In some embodiments, sensing layer 112 may be integrated (e.g. mixed) with the absorbent path, which may be made of a UV transparent and/or visible light transparent material. The absorbent path may have a structure similar to that of a paper or a cloth, or the like. The absorbent path may be a porous material made of polymers.

Sensor 100 may comprise one or more radiation sources or radiation emitters 114 for emitting input radiation 114A (e.g. as schematically illustrated in FIG. 1B). Input radiation 114A may be UV radiation. Radiation source 114 may comprise a UV radiation emitter such as one or more ultraviolet light emitting diodes (UV-LEDs). Radiation source 114 may be optically configured (e.g. by suitable positioning and/or using suitable optical elements such as lenses, reflective materials, mirrors, windows, fiber optics, prisms, etc) to direct radiation toward sensing nodes 106. Radiation source 114 may comprise a plurality of sub-emitters individually configurable to emit radiation of different intensities, wavelengths, etc.

The radiation source 114 (e.g. a UV radiation source) may be optically configured to emit input radiation 114A to irradiate sensing material 106A (e.g. quantum dots). Radiation source 114 (e.g. such as radiation source 114-1) may be optically configured to irradiate sensing material 106A from target side 112A. Radiation source 114 (e.g. such as radiation source 114-2) may be optically configured to irradiate sensing material 106A from the substrate side 112B (e.g. through substrate 108 as shown in FIG. 1B). Radiation source 114 may be optically configured to irradiate sensing material 106A from an edge of sensing layer 112 (e.g. sides other than target side 112A and substrate side 112B). In some embodiments, a vector representative of the principal direction of emission of input radiation 114A of radiation source 114 is parallel to a normal vector of a broad surface of sensing material, 106A sensing layer 112 (e.g. target side 112A or substrate side 112B), and/or substrate 108 (e.g. as would be the case with radiation sources 114-1 and radiation source 114-2 shown in FIG. 1A). A principal radiation direction or principal direction of emission of electromagnetic radiation may be an intensity-weighted average direction of travel of the electromagnetic radiation or may be defined in other ways. In general, electromagnetic radiation may be axially symmetric or may be axially asymmetric about its principal radiation/emission direction. In some embodiments, a vector representative of the principal direction of emission of input radiation 114A of radiation source 114 is non-parallel to a normal vector of a broad surface of sensing material 106A, sensing layer 112, and/or substrate 108 (e.g. as would be the case with radiation source 114-3 shown in FIG. 1A). For example, the vector representative of the principal direction of emission of input radiation 114A of radiation source 114 may intersect the normal vector of a broad surface of sensing material 106A, sensing layer 112, and/or substrate 108 at an angle of at an angle of less than 40°, between approximately 1° and 89° or at an angle of between approximately 20° and 70°, or at an angle of between approximately 35° and 55°.

In some embodiments, optically configuring radiation source 114 to emit input radiation 114A to irradiation sensing material 106A may comprise employing one or more fiber optic elements, prisms, lenses, transparent windows, transparent walls, transparent materials, mirrors and/or other optical elements may be used to direct input radiation 114A from radiation source 114 to sensing material 106A of sensing nodes 106. Such arrangements may offer flexibility in the orientation of radiation source 114 and/or sensing layer 112 and/or sensing material 106A and/or the manner in which analyte 104 is brought into contact with sensing layer 112.

Where sensing layer 112 (including sensing material 106A) is irradiated by radiation source 114 on substrate side 1128, then the target side 112A of sensing layer 112 may be in contact with analyte 104 and/or target molecule(s) 102.

Where substrate 108 is at least partially transparent (e.g. optically transparent), sensing layer 112 (including sensing material 106A) may be irradiated by radiation source 114 from substrate side 1128 of sensing layer 112. This arrangement (having radiation source 114 on substrate side 112B of sensing layer 112) may be advantageous for some applications, as it allows interaction of target side 112A of sensing layer 112 with analyte 104, which is an open side of sensing layer 112 that is not faced or blocked by radiation source 114. Further, this arrangement (having radiation source 114 on substrate side 1128 of sensing layer 112) may be advantageous for some applications, where analyte 104 is not highly transparent, for example where the analyte is blood, as having radiation source 114 on substrate side 1128 of sensing layer 112 allows the activation of sensing material 106A without the input radiation 114A passing through analyte 104.

In some embodiments, sensing material 106A and recognition element 110 may not be integrated and may instead be separated. In some embodiments, sensing material 106A may be applied as a disposable/replaceable sensing layer, or placed on a substrate wherein the substrate and sensing material 106A are disposable. In some embodiments, recognition element 110 may be applied as a disposable/replaceable target molecule recognition layer positioned on sensing layer 112, or placed on a substrate wherein the substrate and recognition element 110 are disposable.

In some embodiments, sensor 100 comprises a detector 128. In some embodiments, sensor 100 may be employed in conjunction with a detector of another device (such as, for example, a mobile computing device as described further herein). Detector 128 may comprise a radiation detector, a light detector, a color detector, an image detector such as a digital image sensor (e.g. a CCD sensor or a CMOS sensor). Detector 128 may be configured to capture at least some of output radiation 11C outputted by sensing nodes 106.

In some embodiments, where the detector (e.g. detector 128 or a detector of a mobile computing device) is on the same side of sensing layer 112 as radiation source 114, a partially reflective material (not shown) may be positioned on the surface of sensing layer 112 for the reflection of the emitted light from the sensing material 106A to help with capturing of the output radiation 114C by the detector. For example, a perforated reflective material may be provided to allow for analyte 104 to still reach to sensing layer 112.

In practice, sensor 100 is employed by contacting sensing layer 112 with analyte 104 and irradiating sensing layer 112 with input radiation 114A. In some embodiments, a specific volume of analyte 104 may be placed on each sensing node 106. In some embodiments, analyte 104 may be kept in contact with sensing nodes 106 for a specific time period (e.g. to reach a stable color changing (nearly equilibrium reaction condition)). In some embodiments, analyte 104 is mixed with a solvent.

In response to input radiation 114A of radiation source 114, sensing material 106A fluoresces (e.g. emits sensing material radiation 114B). For example, when irradiated, the electrons of sensing material 106A (e.g. quantum dots) may be able to accept the UV energy from radiation source 114 and become excited from the valence band to the conduction band. Subsequently, the excited electrons return to the ground state. During the return course, sensing material 106A (e.g. quantum dots) emits fluorescence (e.g. emits sensing material radiation 114B).

In turn, one or more spectral characteristics of sensing material radiation 1146 emitted by the sensing material 106A may be detectably influence-able in response to interaction between recognition element 110 and any target molecule(s) 102 that are present to create output radiation 114C. This may be referred to as loss-of-signal (lower signals at higher concentrations). For example, the presence of target molecule(s) 102 in interaction with recognition element 110 may consume the electrons through photoinduced electron transfer (PET) or resonance energy transfer (RET). These PET and RET phenomena may be enhanced by the presence of recognition element(s) 110. The presence of sites 110A in recognition element(s) 110 may additionally or alternatively enhance the selectivity of sensing layer 112 for detection of one or more specific target molecules 102 in analyte 104. Ultimately, each sensing node 106 emits output radiation 114C (also referred to herein as an output signal) comprising the radiation (e.g. fluorescence) which may be detectably influence-able in response to interaction between the recognition element 110 and target molecule(s) 102 that are present.

The interaction of sensing node 106 with target molecules 102 may generate and/or influences output radiation 114C. Output radiation 114C may by detectable by a detector (e.g. detector 128 or a detector of another device) comprising at least one of: a radiation detector, a light detector, a color detector, an image detector such as a digital image sensor (e.g. a CCD sensor or a CMOS sensor). Spectral characteristics of output radiation 114C represented by signal shape and/or magnitude (e.g. the colour, brightness and/or intensity) may be dependent on one or more of sensing material 106A (e.g. its chemical composition and/or one or more physical attributes thereof), recognition element 110 (e.g. its chemical composition and/or one or more physical attributes thereof) and the chemical composition of target molecule(s) 102 of analyte 104.

In some embodiments, the fluorescence light emission (e.g. sensing material radiation 114B) of sensing material 106A due to input irradiation 114A of radiation source 114 may be quenched or reduced in the presence of a target molecule 102 to create output radiation 114C. For example, a target molecule 102 may absorb the excited state of the emissive electrons. The significance of the signal quenching or reduction may be proportional to the amount or concentration of target molecule 102 in analyte 104.

In some embodiments, the fluorescence light emission (e.g. sensing material radiation 114B) of sensing material 106A due to irradiation by radiation source 114 may be enhanced or increased in the presence of a target molecule 102. For example, target molecule 102 may reduce (through reaction, interaction, etc.) a chemical that absorbs the excited state of the emissive electrons called an active fluorescent quencher (e.g. a rhodamine B derivative). To achieve this, sensing material 106A may be combined with an active quencher. The active fluorescent quencher may absorb light at wavelengths which overlap with the light emission profile of sensing material 106A. Therefore, the fluorescence intensity of sensing material 106A may be reduced by the presence of an active quencher. When target molecules 102 are present, target molecules 102 may alter the structure or composition or the amount of the active quencher, disabling the energy transfer quenching mechanism. Therefore, the increasing concentration of target molecule 102 may enhance the fluorescence intensity emitted by sensing material 106A. The significance of the signal enhancing may be proportional to the amount or concentration of target molecule 102. This may be referred to as gain-of-signal (higher signals at higher concentrations). The gain-of-signal technique may enhance detection range and reduce the detection limit of target molecules.

One or more spectral characteristics of output radiation 114C may be detected/recorded by least one of: a radiation detector, a light detector, a color detector, an image detector such as a digital image sensor (e.g. a CCD sensor or a CMOS sensor), a spectrometer, a radiometer, a fluorescence detector, and/or any kind of portable light detector. Such spectral characteristics include, for example, light intensity (brightness) and spectrum (colour), and/or the changes in the sensor signal, for example, its variation in the light intensity and its spectrum as a result of the quenching (or enhancing). Output radiation 114C may be detected and analyzed by recording red, green, and blue (RGB) values of radiation output 114C. Output radiation 114C may be detected and analyzed by recording cyan, magenta, yellow, and key (CMYK), hue, saturation, brightness (HSB), and HEX values of output radiation 114C. The values of the RGB, and/or CMYK, and/or HSB and/or HEX may be analyzed to identify the quantity or concentration of target molecule(s) 102 in interaction with sensing layer 112. For example, if the peak emission of output radiation 114C of a specific sensing node 106 in the presence of a specific target molecule 102 is in a particular wavelength, the RGB values (or a specific combination of R, G, and B values) related to that wavelength (or near that wavelength) may be used for the assessment of the quantity or concentration of target molecule(s) 102.

In some embodiments, pattern recognition techniques along with data analytics algorithms may be applied to analyze output radiation 114C in terms of target molecule 102 identification and quantification. Such pattern recognition algorithms may use artificial intelligence and/or machine learning and/or deep learning to identify one or more patterns within output radiation 114C. For example, in some embodiments, algorithms may be trained to find patterns in the image signals captured by an image sensor (data sets) to identify and quantify the target materials/molecules (for example biomarkers) of interest in an analyte 104 that interacts with sensing material 106A and/or sensing layer 112. Output radiation 114C may be analyzed during a specific time period (several images at different time intervals to identify the pattern of changing) and/or at a specific time after the interaction of target molecules 102 with the sensing layer 112 (for example, when the change in the image signal is nearly steady state). If a particular target molecule 102 interacts with (e.g. impacts the signal of) more than one sensing node 106, machine learning may be applied to identify and/or quantify the target molecules 102 of interest, based on analyzing the combination of output radiation 114C from sensing nodes 106.

In some embodiments, output radiation 114C from sensing layer 112 may be captured/detected through an optical lens (optional) and by a digital image sensor, such as CCD and CMOS. The lens and the image sensor may be from either a digital camera integrated with the sensor or a stand-alone digital camera, or the digital camera of an electronic device (e.g. camera phone or camera watch).

In some embodiments, analysis of output radiation 114C may be performed by a processor and software (e.g. smartphone processor and a specially designed application, App) to indicate the presence and amount of target molecule(s) 102, for example a biomarker and/or the like in analyte 104. The assessment of the quantity (e.g. concentration in analyte 104) of target molecule(s) 102 may be achieved by utilizing a calibration curve. An image (or other data capture) of output radiation 114C may be captured under specific lighting conditions provided by the sensor design. In some embodiments, the image (or other data capture) of output radiation 114C may be taken under only the lighting or irradiation from the radiation source 114. This may be achieved, for example, by placing sensing material 112 in contact with analyte 114 in a suitable housing (e.g. housing 216 discussed further herein). Such a housing may be light-tight (to control the light admitted into an interior of the housing and/or to particular regions within the interior of the housing) or partially light-tight. This may be advantageous as the fluorescence lighting color and intensity of output radiation 114C may not be affected by the environmental lighting and sensor 100 may be calibrated at standard conditions for any digital camera and its processor (e.g. camera of a mobile computing device). Sensor 100 may be advantageous as compared to some other sensors, as it does not rely on some means of measuring sensor signals from a component connected to sensing layer 112 (such as electrodes, for example) and can operate with a mobile computing device such as, for example, a camera phone, a laptop, a smartphone, a tablet computing device, a smart watch or a smart wearable device, which are widely available.

In some embodiments, the intensity and/or wavelength of input radiation 114A may be varied when sensing layer 112 is in contact with analyte 104. For example, sensing layer 112 may be excited at different intensities and/or wavelengths to generate various signals. By activating sensing layer 112 at different intensities (UV radiant power) and/or at different wavelengths (UV photon energy) over suitable period(s) of time, a response curve (different colors and intensities of output radiation 114C emitted from sensing layer 112, for various wavelengths and/or intensities of radiation source 114) may be generated. Such response curves may be analyzed to identify the presence and/or the amount of target molecule(s) 102. This approach of varying radiation intensity and/or wavelength of input radiation 114A may be advantageous for generating multiple signals, compared to other prior art methods because the intensity and/or wavelength of radiation source 114 (e.g. UV-LEDs) can be easily (e.g. precisely and quickly) altered. The approach of varying radiation intensity and/or wavelength of input radiation 114A to generate response curves may enhance the sensor 100 performance relative to prior art techniques using a specific intensity and/or wavelength, for example, by improving selectivity and/or sensitivity of the sensing layer 112 to particular chemicals. Further, the approach of varying radiation intensity and/or wavelength of radiation 114A to generate response curves may enable multiplexed measurement of several target molecules 102, for example biomarkers, without the need of using a particular sensing material 106A and/or recognition element 110 for each of target molecules 102 and instead by interpreting output radiation 114C generated by different sensing nodes 106 that have been irradiated at different intensities and wavelengths. Because a sensing layer 112 may respond differently to different target molecules 102 when the radiant power (intensity) or photon energy (wavelength) changes, such differences in the responses may be analyzed (for example by a program or software) to detect and quantify target molecule(s) 102.

Sensor 100 may comprise a single sensing node 106. Sensor 100 may comprise multiple identical sensing nodes 106 (e.g. sensing nodes having the same sensing material 106A and the same recognition element 110). Sensor 100 may comprise multiple different sensing nodes 106. Sensing nodes 106 may vary by varying sensing material 106A and/or by varying recognition element 110. Sensing material 106A may differ in amount, concentration (e.g. relative to recognition element 110), shape, size and/or composition. Recognition element 110 may differ in amount, concentration (e.g. relative to sensing material 106A) composition or in type or number of recognition sites 110A. For example, sensor 100 may comprise at least two sensing nodes 106 comprising the same sensing material 106A and different recognition element 110. As another example, sensor 100 may comprise at least two sensing nodes 106 comprising different sensing materials 106A and the same recognition element 110. As another example, sensor 100 may comprise at least two sensing nodes 106 comprising different sensing materials 106A and different recognition element 110).

In some embodiments, sensing layer 112, may be optimized for enhanced sensing of a particular analyte 104 and/or particular target molecule(s) 102. In some embodiments, sensing layer 112 may comprise sensing nodes 106 with the same sensing material 106A composition and same recognition element 110, but at different ratios, and quantities of sensing material 106A and recognition element 110. This may be advantages because although sensing material 106A and recognition element 110 are the same (e.g. for targeting a single type of target molecule 102), each particular combination may be applied (optimized) for detecting a specific concentration range or quantity of that target molecule 102.

Where at least two different sensing nodes 106 are provided, a combination of distinct output radiations 114C may be generated (e.g. one output radiation for each unique sensing node 106), depending on the presence and amounts of various target molecules 102 interacting with the different sensing nodes 106. In some embodiments, each sensing node 106 is configured for a different target molecule 102. By analyzing output radiation 114C from each sensing node 106, the presence and amounts of multiple target molecules 102 of interest may be assessed. Such a system of several sensing nodes 106 may enable multiplexed measurement of several target molecules 102 more accurately. Such a system of several sensing nodes 106 may also enable multiplexed measurement of several target molecules 102. Such measurement, in some embodiments, may be achieved without using a different recognition element 110 or type of recognition site 110A on each sensing node 106, but instead by interpreting output radiation 114C generated by different sensing nodes 106 comprising different sensing materials 106A.

In some embodiments, the analysis of output radiation 114C may be performed by utilizing machine/deep learning algorithms (e.g. algorithms trained to find patterns in data sets), to assess the presence and amounts of target molecules 102. A training model may be applied to teach the algorithms that interpret output radiation 114C (for example, software that converts output radiation 114C to identify the presence and/or amount of target molecules 102) how to discriminate among different target molecules 102. A different training model may additionally or alternatively be applied to estimate the amount of different target molecules 102. The training model to teach the system, to discriminate for, or to detect the quantity of a particular target molecule 102 may be initiated with a limited number of data points (sensor response patterns) from artificial samples (e.g. artificial biofluid samples or biofluid samples from volunteers). The model may then be enhanced over time by collecting more data points from users, which may be collected through a suitable computing device application (e.g. mobile computing device-based). output radiation 114C received by the detector (e.g. detector 128) may be transferred through such an application to be analyzed in a central processing platform.

Sensor 100 may be provided with a plurality of sensing nodes 106, where one or more of those sensing nodes may act as reference nodes. The reference node may comprise a sensing node 106 to be irradiated by input radiation 114A in the absence of any analyte 104 or to be irradiated by input radiation 114A in the presence of a blank analyte (analyte 104 without any target molecule 102). Alternatively or additionally, particular sensing material 106A (e.g. particular material or size or shape or a combination of these which is different from the target-specific emissive quantum dots) chosen such that its excited electrons are not significantly absorbed by target molecules 102 may be applied to act as the reference node that is not affected by the ambient environment and/or analyte 104 and/or target molecules 102. The output radiation 114C from these reference sensing nodes may be used to normalize (correct the background of, or the noise of) output radiation 114C (e.g. color or intensity) of the active sensing nodes 106 for a more accurate detection and quantification with sensor 100.

In some embodiments, sensor 100 (and/or one or more sensing nodes 106 thereof) may be calibrated by contacting one or more analytes having known concentrations of target molecules 102 with sensing layer 112. For example, this may be result in a calibration curve for sensor 100 which may be applied to determine the amount of a target molecule 102 based on output of sensor 100. In some embodiments, it may be beneficial (for measurement purposes) to provide a specific or desired amount of analyte in contact with sensing layer 112. In some embodiments, provision of a specific or desired amount of analyte may be achieved by dropping specific volumes of analyte 104 on sensing layer 112. In some embodiments, this may be achieved by contacting sensing layer 112 with analyte 104 for a specific time period. For example, this may be achieved by implementing a function to control the time where sensing layer 112 is in contact with analyte 104 or by implementing a timer to become activated upon the contact of analyte 104 with sensing layer 112 and trigger the output radiation 114C, for example taking the image of sensing layer 112 after a specific time period or at set time intervals.

In some embodiments, sensor 100 may comprise a sub-sensor to monitor radiation of radiation source 114. For example, a UV sensor may be implemented to monitor input radiation 114A of radiation source 114. This may allow for making corrections for any dependence of the output of sensor 100 based on radiation 114A of radiation source 114.

In some embodiments, sensor 100 may comprise one or more repellant modules for selectively repelling target molecule(s) 102 that are interacting with (e.g. bound to) sensing layer 112 so that, for example, sensing layer 112 may be re-used. The repellant module may comprise a pair of electrodes, laser-engraved graphene (LEG) and/or redox-active nanoreporters (RARs). This combination may offer the advantage of providing the sensing nodes with the ability to be regenerated in situ by selectively applying constant potential to the working electrode, which repels the bound target molecule(s) 102 from sensing layer 112, for re-usability.

In some embodiments, sensor 100 may comprise a release or stimulating module for selectively stimulating the release of biofluid analyte 104 from skin, such as an iontophoresis module or heating module. This release or stimulating module may offer the advantage of providing the sensor with biofluids such as sweat from the skin which may be in contact with sensor 100.

In some embodiments, sensor 100 is manufactured using the same or similar fabrication processes typically employed for fabricating an ultraviolet light emitting diode (UV-LED) chip or UV-LED wafer by adding sensing layer 112 to the LED fabrication process (e.g. on top of the LED, the LED chip or the LED package).

In some embodiments, a plurality of sensing nodes 106 are provided on a roll of material (e.g. coated, immobilized or printed on a roll of material) such as a paper or polymer roll. In practice, the roll can be advanced (manually or automatically), so that at different time intervals unexposed (e.g. unused) sensing nodes 106 may come in contact with analyte 104. This configuration may be employed for automated remote monitoring. For example, by unrolling the roll of sensing nodes 106 so as to expose fresh sensing nodes 106 to water, the water quality at specific time intervals may be monitored (e.g. by taking images of the submerged sensing nodes 106 to capture output radiation 114C). Such images may be transmitted wirelessly such that this system and method may be automated and/or remotely controlled.

In some embodiments, sensor 100 is mountable to a digital camera. In some embodiments, sensor 100 is mountable to a mobile computing device, for example in front of the camera lens of a cell phone tablet computing device, or smartphone. In some embodiments, sensor 100 is mountable to a digital watch for example at the back or front of an Apple™ watch. In some embodiments, the digital watch comprises a detector (e.g. light detector). In some embodiments, the digital watch comprises a lens and an image sensor (e.g. a CCD or CMOS sensor). Such mountable feature may be advantageous as some already available sensors and devices can be used as a platform for signal measurement.

Further embodiments of sensors and/or their components described herein may use similar reference numerals (e.g. with a preceding digit, a trailing symbol, a trailing letter and/or a trailing number) to those used to describe sensor 100 and/or its components. Unless the context or description dictates otherwise, such sensors and/or their components may exhibit features and/or characteristics and/or may function in a manner which may be similar to the features and characteristics and function of sensor 100 and/or its components (or vice versa). For example, sensors/devices 200-1100 described in more detail below are sensors according to particular embodiments of the invention. Unless the context or description dictates otherwise, sensors/devices 200-1100 may have features and/or characteristics similar to those discussed herein for sensor 100 (or vice versa). As another example, radiation sources 212-1112 described in more detail below are radiation sources according to particular embodiments of the invention. Unless the context or description dictates otherwise, radiation sources 212-1112 may have features and/or characteristics similar to those discussed herein for radiation emitter 112. Further, unless the context or description dictates otherwise, it should also be understood that when referring to features and/or characteristics of sensor 100 and/or its components, the corresponding description should be understood to apply to any of the particular embodiments of sensors, devices and/or their components.

Another aspect of the invention provides a device for detection or quantitative measurement of a first target molecule in an analyte using the camera of a mobile computing device such as, for example, a camera phone, a smartphone, a tablet computing device, a smart watch or a smart wearable device. The device may comprise a radiation emitter supported in a housing and controllable to emit input radiation and a solid-phase substrate comprising a first sensing node provided in solid phase on the substrate for exposure to the analyte. The sensing node may comprise a first radiation-activatable fluorescence material and a first recognition element for interaction with the first target molecule in the analyte. The solid-phase substrate may be insertable into the housing in a location where the input radiation impinges on the first sensing node. The radiation-activatable fluorescence material may be fluoresce-able in response to interaction with the input radiation to thereby cause the first sensing node to emit output radiation. One or more spectral characteristics of the output radiation may be detectably influence-able in response to interaction between the first recognition element and the first target molecule. The device may be mountable, or otherwise locatable, relative to the camera of the mobile computing device such that at least some of the output radiation exits the housing through an aperture and is detectable by the camera.

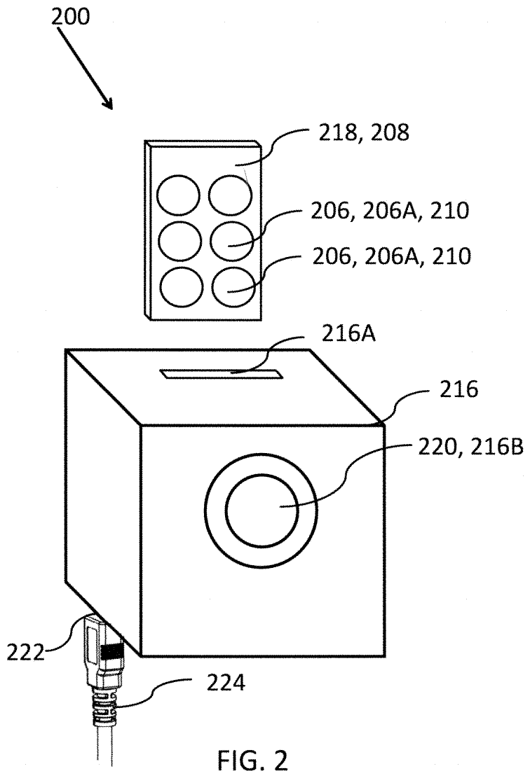

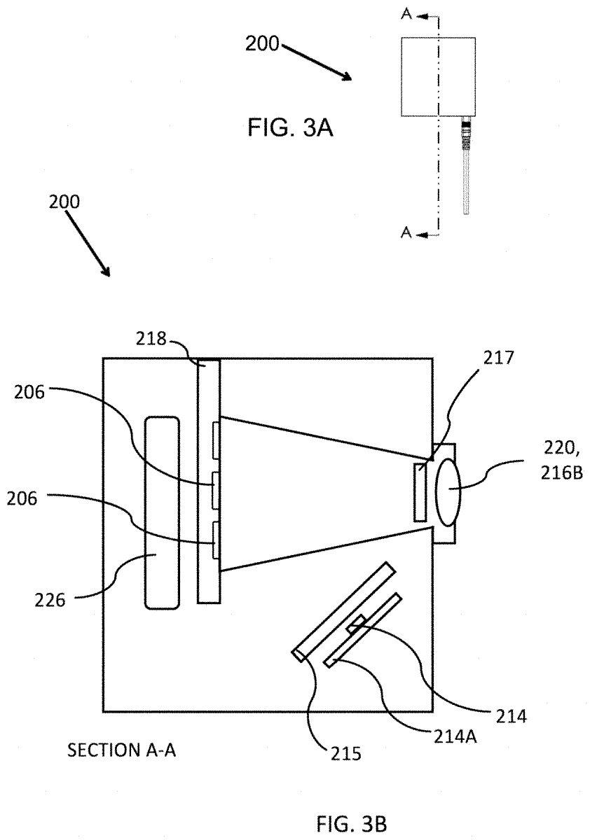

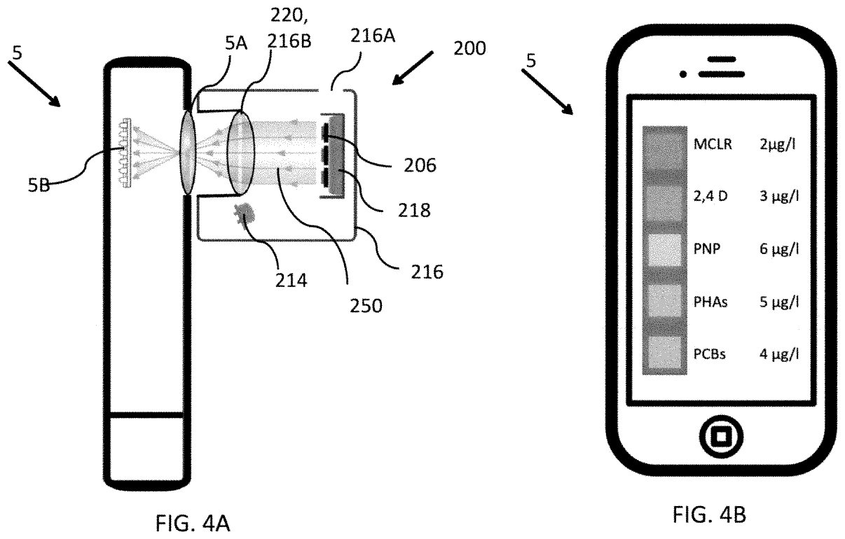

FIG. 2 depicts an exemplary device 200 for detection or quantitative measurement of a target molecule in an analyte using the camera of a mobile computing device 5 according to an example embodiment of the invention. FIG. 3A depicts a side view of device 200 and FIG. 3B depicts a cross-sectional view of device 200 taken from section A-A (shown in FIG. 3A). FIG. 4A is a schematic cross section of device 200 mounted on mobile computing device 5.

Mobile computing device 5 may comprise any suitable mobile computing device such as, for example, a digital camera, a camera phone, a smartphone, a tablet computing device, a smart watch or a smart wearable device. The camera of mobile computing device may comprise an optical lens 5A and an image sensor 5B (e.g. a CCD sensor or a CMOS sensor).

Device 200 comprises a radiation emitter or radiation source 214 (e.g. substantially similar to radiation source 114) supported in a housing 216 and controllable to emit input radiation. Radiation source 214 may be mounted or attached to a printed circuit board (PCB) 214A. Optionally, a UV pass filter 215 for blocking visible lights emitted from the UV-LED may be provided. Optionally, a filter 217 may be provided to block or reduce UV radiation (and/or other wavelengths of radiation) emitted from radiation source 214. Filter 217 may comprise, for example, a visible light band pass filter or a visible light pass filter.

In some embodiments, radiation source 214 may be positioned to direct input radiation, wherein its principal (central) radiation emission direction is substantially directly facing sensing nodes 206. For example, radiation source 214 may be positioned under, over, or beside aperture 216B, where its radiation emission side is facing sensing nodes 206. In some embodiments, a vector of the principal emission direction of input radiation of radiation source 214 is parallel to one or more of: a normal vector from the surface of the transparent portion 2166 of housing 216 and/or lens 220 and a normal vector from the a broad surface of sensor card 218 and/or sensing nodes 206. In some embodiments, a vector of the principal emission direction of input radiation of radiation source 214 is non-parallel to one or more of: a normal vector from the surface of the transparent portion 216B of housing 216 and/or lens 220 and a normal vector from the a broad surface of sensor card 218 and/or sensing nodes 206. For example, the vector representative of the principal direction of emission of radiation of radiation source 214 may intersect one or more of: a normal vector from the surface of the transparent portion 2166 of housing 216 and/or lens 220 and a normal vector from the a broad surface of sensor card 218 and/or sensing nodes 206 at an angle of less than 40°, between approximately 1° and 89° or at an angle of between approximately 20° and 70°, or at an angle of between approximately 35° and 55°.

A sensor card 218 may be insertable into housing 216. Sensor card 218 may comprise a solid-phase substrate 208 (e.g. substantially similar to substrate 108) comprising one or more sensing nodes 206 (e.g. substantially similar to sensing nodes 106) provided in solid phase on substrate 208 for exposure to a sample of an analyte (e.g. analyte 104). Sensor card 218 may be insertable into a slot 216A of housing 216 in a location where the input radiation from radiation source 214 impinges on the one or more sensing nodes 206 of sensor card 218. In the illustrated embodiment, sensor card 218 comprises six sensing nodes 206 but this is not mandatory and sensor card 218 may comprise any suitable number of sensing nodes 206.

Like sensing nodes 106, sensing nodes 206 may comprise a radiation-activatable fluorescence material 206A (e.g. substantially similar to sensing material 106A and also referred to herein as sensing material 206A) and recognition element 210 (e.g. substantially similar to recognition element 110) for interaction with target molecule(s) 102 in the sample (e.g. analyte 104). Sensing material 206A may be fluoresceable in response to interaction with the input radiation of radiation source 214 to thereby cause the sensing node(s) 206 to emit output radiation. One or more spectral characteristics of the output radiation may be detectably influence-able in response to interaction between first recognition element 210 and the target molecule(s) 102. Device 200 may be mountable, or otherwise locatable, relative to the camera of mobile computing device 5 such that at least some of the output radiation exits housing 216 through an aperture 216B defined by housing 216 and is detectable by the camera. Aperture 216B may comprise an optically transparent portion of housing 216 or may comprise an optical lens 220. An example of the possible light streamlines from sensing nodes 206 to image sensor 5B of mobile computing device 5 are schematically illustrated by arrows 250 in FIG. 4A.

Housing 216 may be mountable, or otherwise locatable, relative to the camera of mobile computing device 5 such that a lens 5A and/or an image sensor 5B of mobile computing device 5 is aligned with aperture 216B such that at least some of the output radiation exits housing 216 through an aperture 216B defined by housing 216 and is detectable by the camera of mobile computing device 5. Housing 216 may be attachable or otherwise locatable relative to the camera of mobile computing device 5 by any suitable means such as, for example, a clip, an adhesive, a clamp, etc. In some embodiments, a case or cover for mobile computing device 5 is attached to, integral with or attachable to housing 216.

Device 200 may comprise a battery 226 for powering radiation source 214 and/or any other components of device 200. Battery 226 may be replaceable. Battery 226 may be rechargeable. For example, device 200 may comprise a power slot 222 (e.g. micro-USB, mini-USB, UBC C, USB A, a barrel power connector, etc.) for receiving a charging cable 224. Alternatively, device 200 may be powered by mobile computing device 5 (e.g. by wired or wireless connection).

Once device 200 is installed on the camera of mobile computing device 5, the camera may capture images of output radiation of the sensing nodes 206 while the sensing nodes 206 are excited by radiation from radiation source 214 (e.g. as described in relation to sensor 100), through transparent section 216B of the housing 216 and/or through optical lens 220. The images captured by the camera of mobile computing device 5 may be analyzed by a software (e.g. a software application of mobile computing device 5) and spectral characteristics (e.g. colour and/or brightness) of output radiation in the images may be analyzed by the software to identify and/or quantify target molecules 102 in an analyte 104 contacted with sensing nodes 206. For example, the RGB value of output radiation of each sensing node 206 may be captured by the camera of mobile computing device 5 and analyzed by the software to identify the concentration of target molecule 102 using a specified conversion formula or a calibration curve, for example. In some embodiments, the analysis may be done natively by software of mobile computing device 5 or the images may be uploaded to a network (e.g. a cloud computing network) for analysis remotely.

Results of analysis of the output radiation of sensing nodes 6 within device 200 captured by mobile computing device 5 may be displayed on a display of mobile computing device. For example, FIG. 4B is a schematic depiction of a mobile computing device displaying results of analysis of an image of one or more sensing nodes 6 configured to detect various target molecules 2 (e.g. MCLR, 2, 4 D, PNP, PHAs, PCBs).

In some embodiment, sensor 100 is combined with a wearable device, for example a wristband. The wearable device may comprise a lens, an image sensor, a processor, and a user interface or display. In some embodiments, the image sensor or the light detector may be sensitive to only a particular wavelength, for example the wavelength range associated with the blue light.

Another aspect of the invention provides a wearable device for detection or quantitative measurement of a first target molecule in an analyte. The wearable device comprises a radiation emitter supported in a wearable housing and controllable to emit input radiation, an image sensor supported in the wearable housing, a substrate comprising a first sensing node provided on the substrate and an optical system for directing input radiation onto the first sensing node. The sensing node comprises a first radiation-activatable fluorescence material and a first recognition element for interaction with the first target molecule in the analyte. The substrate is mountable to an exterior of the housing for exposure to the sample when the apparatus is in use. The first radiation-activatable fluorescence material is fluoresce-able in response to interaction with the input radiation to thereby cause the first sensing node to emit output radiation. One or more spectral characteristics of the output radiation may be detectably influence-able in response to interaction between the first recognition element and the first target molecule. At least some of the output radiation is detectable by the image sensor.

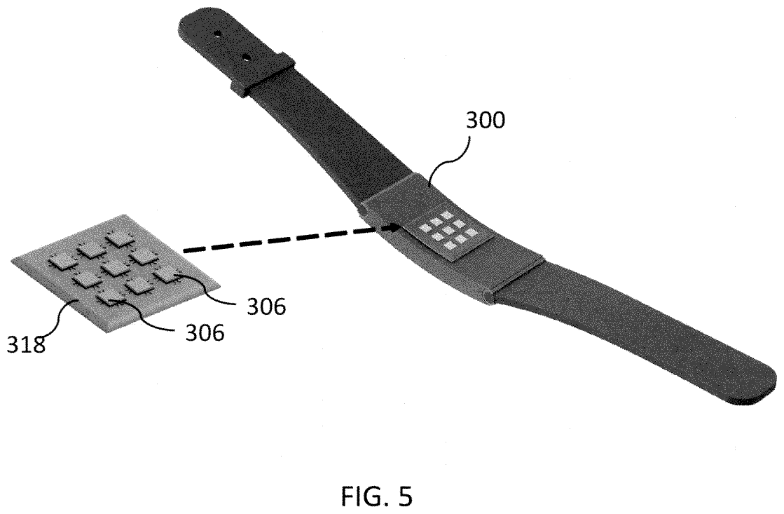

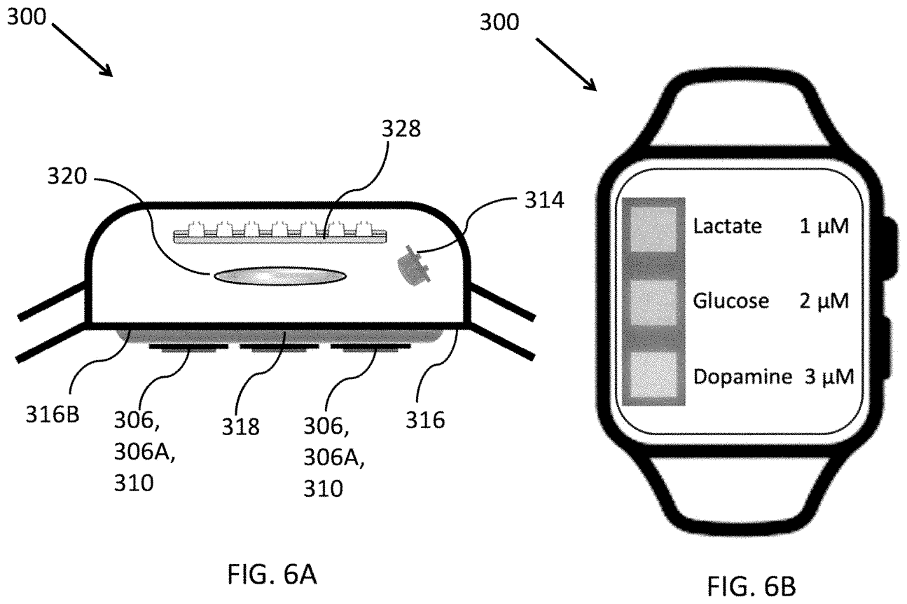

FIG. 5 depicts an exemplary wearable device 300 for detection or quantitative measurement of a target molecule in an analyte 104 according to an example embodiment of the invention. FIG. 6A depicts a schematic cross-sectional side view of wearable device 300 and FIG. 6B depicts a schematic view of an optional display of wearable device 300.

Wearable device 300 may comprise, for example, a wristband, a smartwatch, an ankle band, a chest mounted device (e.g. substantially similar to some heart rate monitor sensors), a ring to be worn on a finger, etc.

A sensor card 318 may be attachable to (adherable to, insertable in, mountable to, etc.) housing 316. For example, housing 316 may comprise a slot for receiving sensor card 318. Sensor card 318 may be permanently attached to housing 316. Sensor cards 318 may be disposable and releasably attached to housing 316 such that a new sensor card 318 can replace a used or otherwise undesired sensor card 318 of wearable device 300. Sensor card 318 may comprise a solid-phase substrate (e.g. substantially similar to substrate 108) comprising one or more sensing nodes 306 (e.g. substantially similar to sensing nodes 106) provided in solid phase on the substrate for exposure to a sample of an analyte (e.g. analyte 104). Sensor card 318 may be attachable to housing 316 at a location where the input radiation from radiation source 314 impinges on the one or more sensing nodes 306 of sensor card 318. In the illustrated embodiment, sensor card 318 comprises nine sensing nodes 306 but this is not mandatory and sensor card 318 may comprise any suitable number of sensing nodes 306. A disposable membrane (not shown) may be applied to cover one or more sensing nodes 306.

Like sensing nodes 106, sensing nodes 306 may comprise a radiation-activatable fluorescence material 306A (e.g. substantially similar to sensing material 106A and also referred to herein as sensing material 306A) and recognition element 310 (e.g. substantially similar to recognition element 110) for interaction with target molecule(s) 102 in the sample (e.g. analyte 104). Sensing material 306A may be fluoresceable in response to interaction with the input radiation of a radiation source 314 to thereby cause the sensing node(s) 306 to emit output radiation. One or more spectral characteristics of the output radiation may be detectably influence-able in response to interaction between recognition element 310 and target molecule(s) 102.

Wearable device 300 comprises a radiation emitter or radiation source 314 (e.g. substantially similar to radiation source 114) supported in a housing 316 and controllable to emit input radiation. Radiation source 314 may be mounted or attached to a printed circuit board (PCB). Optionally, a UV pass filter for blocking visible lights emitted from the UV-LED may be provided. In some embodiments, radiation from radiation source 314 is directed to sensing nodes 306 through an optical system comprising one or more fiber optic elements, by mirrors, prisms, lenses and/or other optical elements. In some embodiments, at least some of the output radiation is directed toward sensing nodes 306 through an aperture 316B defined by housing 316. Aperture 316B may comprise an optically transparent portion of housing 316, one or more mirrors, one or more lenses, fiber optic elements, etc.

In some embodiments, radiation source 314 may be positioned to direct radiation, wherein its principal (central) radiation emission direction is substantially directly facing sensing nodes 306 (discussed further herein). For example, radiation source 314 may be positioned in relation to (e.g. adjacent to) aperture 316B, where its radiation emission side is facing the sensing nodes 306. In some embodiments, a vector of the principal emission direction of radiation source 314 is parallel to one or more of: a normal vector from the surface of the transparent portion 316B of housing 316 and/or lens 320 and a normal vector from the a broad surface of sensor card 318 and/or sensing nodes 306. In some embodiments, a vector of the principal emission direction of radiation source 314 is non-parallel to one or more of: a normal vector from the surface of the transparent portion 316B of housing 316 and/or lens 320 and a normal vector from the a broad surface of sensor card 318 and/or sensing nodes 306. For example, the vector representative of the principal direction of emission of radiation of radiation source 314 may intersect one or more of: a normal vector from the surface of the transparent portion 316B of housing 316 and/or lens 320 and a normal vector from the a broad surface of sensor card 318 and/or sensing nodes 306 at an angle of less than 40°, between approximately 1° and 89° or at an angle of between approximately 30° and 70°, or at an angle of between approximately 35° and 55°.

Wearable device 300 may comprise a detector 328 within housing 316. Detector 328 may comprise a radiation detector, a light detector, a color detector, an image detector such as a digital image sensor (e.g. a CCD sensor or a CMOS sensor), a spectrometer, a radiometer, a fluorescence detector, and/or any kind of portable light detector.

Wearable device 300 may comprise a battery for powering radiation source 314, detector 328 and/or any other components of wearable device 300. The battery may be replaceable. The battery may be rechargeable. For example, wearable device 300 may comprise a power slot (e.g. micro-USB, mini-USB, UBC C, USB A, a barrel power connector, etc.) for receiving a charging cable.

When wearable device 300 is worn, sensing nodes 306 may be in direct or indirect contact with the skin of the user and sensing nodes 306 may be exposed to biofluid analyte 104 of the user (e.g. sweat generated on the skin or interstitial fluid on the skin) for example, through a patch. When sensing nodes 306 are exposed to analyte 104, one or more spectral characteristics of the sensing material radiation emitted by the sensing material 306 may be detectably influenced by interaction between recognition element 310 and any target molecule(s) 102 that are present in analyte 304. The resultant output radiation of sensing nodes 306 may be detected by detector 328 and may be analyzed (e.g. by software executable by wearable device, or remotely located as described further herein) to indicate the presence or quantity (e.g. concentration) of target molecule(s) 102. For example, device 300 may output data captured by detector 328 to a mobile computing device, a cloud-based network, a personal computing device, etc. by wired or wireless connection.

In some embodiments, wearable device 300 comprises a display wherein the concentration of several target molecules 102 such as glucose, lactate, and dopamine are displayed as shown, for example, in FIG. 6B.

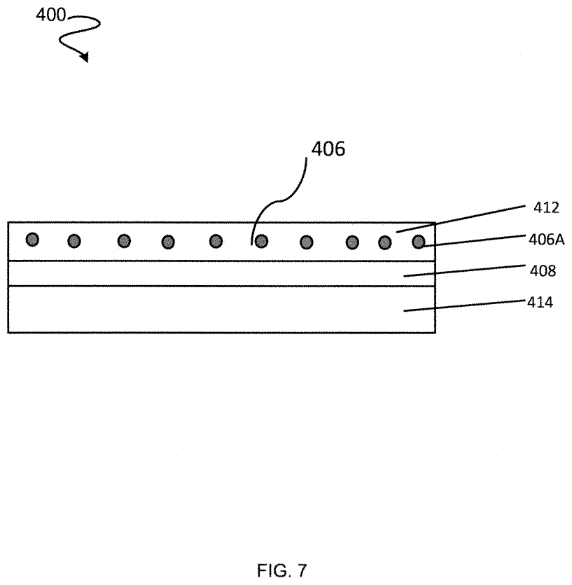

FIG. 7 depicts a sensor device 400 according to an example embodiment of the invention. Sensor device 400 may be substantially similar to sensor 100. Sensor device 400 comprises a radiation source 414 (e.g. substantially similar to radiation source 114) configured to emit input radiation to irradiate a sensing node 406 (e.g. substantially similar to sensing nodes 106) of sensing layer 412 immobilized on a radiation transparent substrate (radiation transparent material) 408 (e.g. substantially similar to substrate 108), for example quartz or transparent polymer. Sensing node 406 comprises quantum dots as sensing material 406A (e.g. substantially similar to sensing material 106A) of, for example, zinc oxide (ZnO) nano-particles or cadmium sulfide (CdS), cadmium selenide (CdSe), zinc selenide (ZnSe), indium phosphide (InP), carbon quantum dots, graphene quantum dots, or a combination of these that are configured to receive radiation from the radiation source 414 and emit photons, for example in the form of fluorescent light.

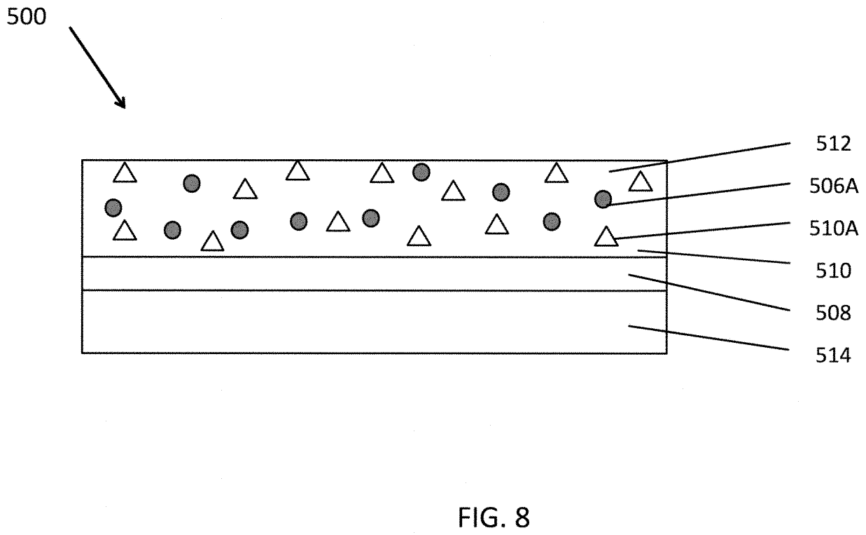

FIG. 8 depicts a sensor device 500 according to an example embodiment of the invention. Sensor device 500 may be substantially similar to sensor 100. Sensor device 500 comprises a radiation source 514 (e.g. substantially similar to radiation source 114) configured to emit input radiation to irradiate a sensing layer 512 immobilized on a radiation transparent substrate 508. Sensing layer 512 may comprises functional monomer, for example 3-Aminopropyltriethoxysilane (APTES) or 5-indolyl boronic acid that may form a polymer recognition element 510. Sensing layer comprising quantum dots as sensing material 506A (e.g. substantially similar to sensing material 106A) configured to receive radiation from the radiation source 514 and imprinted sites 510A that are cavities in the recognition element 510 with an affinity for a chosen template molecule, configured to interact with a target molecule 102 in an analyte 104 to influence one or more spectral characteristics of the output radiation of sensing layer 512.

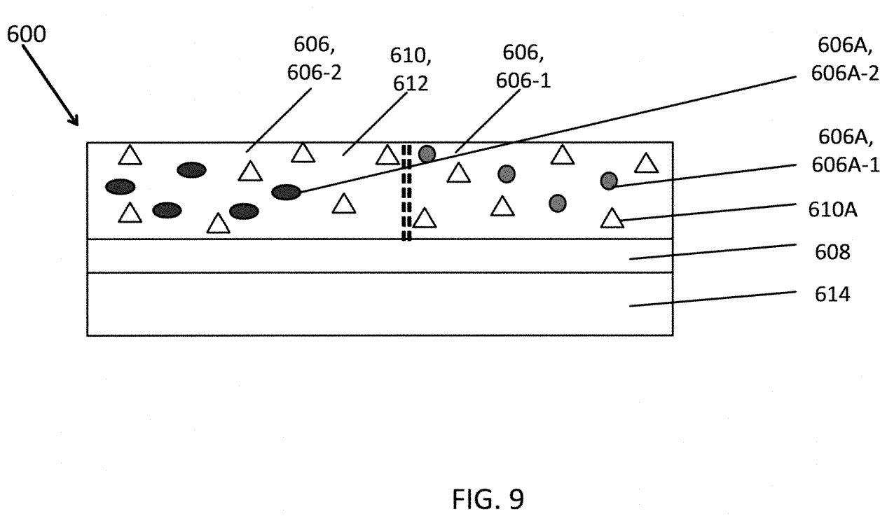

FIG. 9 depicts a sensor device 600 according to an example embodiment of the invention. Sensor device 600 may be substantially similar to sensor 100. Sensor device 600 comprises a radiation source 614 (e.g. substantially similar to radiation source 114) configured to emit input radiation to irradiate one or more sensing nodes 606 of a sensing layer 612 immobilized on a radiation transparent substrate 608. Sensing layer 612 comprises a first sensing node 606-1 comprising a sensing material 606A of a first type of quantum dots 606A-1 and imprinted sites 610A in recognition element 610. Sensing layer 612 comprises a second sensing node 606-2 comprising a sensing material 606A of a second type of quantum dots 606A-2 and imprinted sites 610A in recognition element 610. This configuration may be advantage for capturing the images of lights of different wavelengths, and likely at different time of light transition that may help with more accurate analysis of the presence and/or quantity of target molecule(s) 102 in analyte 104.

In some embodiments, the first type of quantum dots 606A-1 is different from the second type of quantum dots 606A-2 (e.g. in material, shape, size or any combination of these). In some embodiments, the first type of quantum dots 606A-1 are target-specific emissive quantum dots while the second type of quantum dots 606A-2 emit radiation that is not significantly influenced by target molecule(s) 102 and/or analyte 104 such that the second type of quantum dots 606A-2 may be employed as a reference emission that is not affected by the ambient environment while radiation outputted by the target-specific emissive quantum dots of the first type 606A-1 is influenced by target molecule(s) 102 and/or analyte 104. This configuration may be advantageous for capturing the images of lights of different wavelengths and using one (second type of quantum dots 606A-2) as a reference, that may help with more accurate analysis of the presence and/or quantity of target molecule(s) 102.

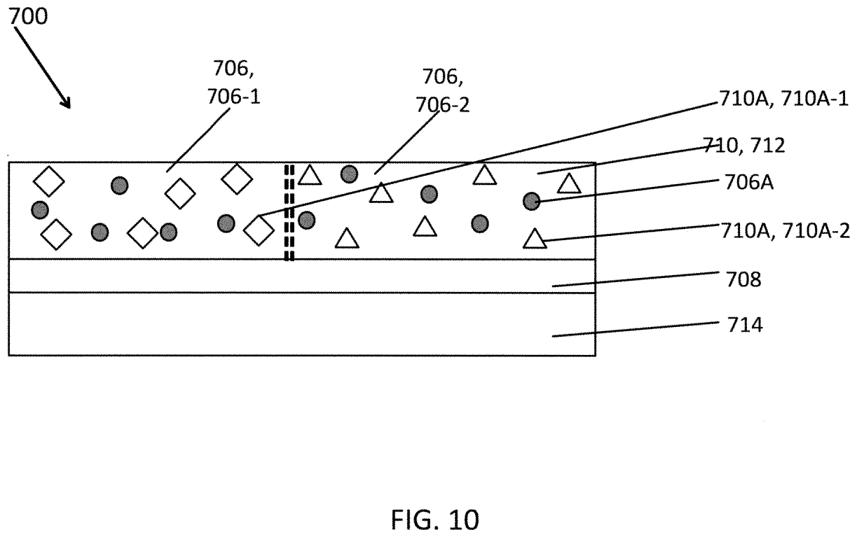

FIG. 10 depicts a sensor device 700 according to an example embodiment of the invention. Sensor device 700 may be substantially similar to sensor 100. Sensor device 700 comprises a radiation source 714 (e.g. substantially similar to radiation source 114) configured to emit input radiation to irradiate one or more sensing nodes 706 of a sensing layer 712 immobilized on a radiation transparent substrate 708. Sensing layer 712 comprises a first sensing node 706-1 comprising a sensing material 706A of quantum dots and a first type of imprinted sites 710A-1 in recognition element 710 and a second sensing node 706-2 comprising a sensing material 706A of quantum dots and a second type of imprinted sites 710A-2 in recognition element 710. In some embodiments, first type of imprinted sites 710A-1 have an affinity for a first type of target molecule 102 while second type of imprinted sites 710A-2 have an affinity for a second type of target molecule 102. First type of imprinted sites 710A-1 may be generally arranged in a first sensing node 706-1 of sensing layer 712 while second type of imprinted sites 710A-2 may be generally arranged in a different second sensing node 706-2 of sensing layer 712, as depicted. In some embodiments, sensing material 706A in the first sensing node 706-1 of sensing layer 712 is similar or identical to sensing material 706A in the second sensing node 706-2 of sensing layer 712. In some embodiments, sensing material 706A in the first sensing node 706-1 of sensing layer 712 is different (e.g. the quantum dots are of a different composition, size and/or shape) to sensing material 706A in the second sensing node 706-2 of sensing layer 712. This configuration may be advantageous for analysis of the presence and/or quantity of multiple types of target molecules 102 in analyte 104.

In some embodiments, a single type of sensing material 706A is provided for multiple sensing nodes 706 each having a different type of imprinted sites 710A. In some embodiments, a different sensing material 706A (e.g. quantum dots of a different composition, size and/or shape) is provided for each sensing node 706 having a different type of imprinted sites 710A. In some embodiments, sensing nodes 706 having different sensing materials 706A (e.g. quantum dots of a different composition, size and/or shape) are provided with the same type of imprinted sites 710A. In some embodiments, in a first sensing node 706-1, the first type of quantum dots 706A-1 are integrated with target-specific recognition element 710 with imprinted sites 710A, while in the second sensing node 706-2 the first type of quantum dots 706A-1 are integrated with imprinted polymer without any imprinted sites 710A (non-imprinted polymer—NIP) so that the second sensing node 706-2 is not significantly influenced by target molecule(s) 102. In some such embodiments, radiation output from second sensing node 706-2 may be employed as a reference radiation profile (which, for example, may not be significantly affected by the presence of target molecule 102) while radiation output by the target-specific first sensing node 706-1 is influenced by target molecule(s) 102. Using a sensing node (e.g. sensing node 706-2) to provide a reference radiation profile may be advantageous for obtaining a more accurate detection and/or quantification of a target molecule.

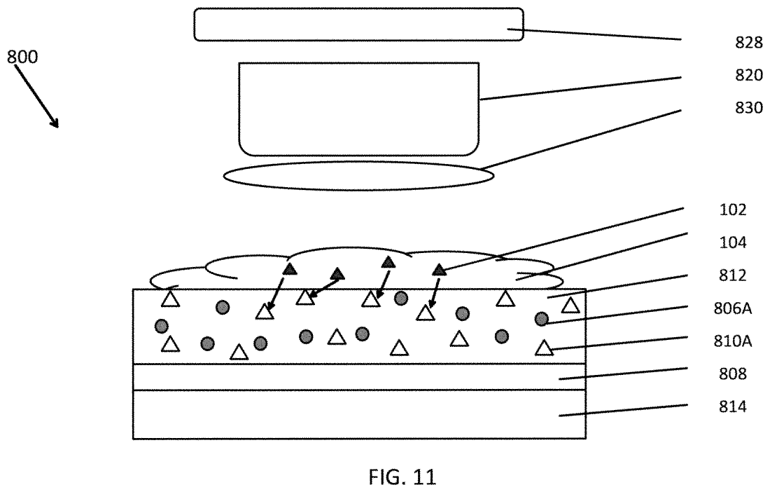

FIG. 11 depicts a sensor device 800 according to an example embodiment of the invention. Sensor device 800 may be substantially similar to sensor 100. Sensor device 800 comprises a radiation source 814 (e.g. substantially similar to radiation source 114) configured to emit input radiation to irradiate a sensing layer 812 immobilized on a radiation transparent substrate 808 (e.g. substantially similar to substrate 108), a detector 828 (e.g. substantially similar to detector 128) and an optional lens 820. Detector 828 may comprise a spectrometer or other types of light detector, and in such case, the lens 820 may be optional or may not be required. Sensing layer 812 comprises sensing material 806A of quantum dots configured to receive input radiation from radiation source 814. Sensing layer 812 also comprises imprinted sites 810A that are configured to selectively interact with (e.g. any form of physical or chemical interactions or binding or reaction, and the like) target molecule(s) 102. When analyte 104 containing target molecule(s) 102 is brought into contact with sensing layer 812, target molecule(s) 102 may bind to the imprinted sites 810A with high affinity and specificity.

When the sensing material 806A is irradiated by the radiation source 814, electrons in the quantum dots may be excited to a state of higher energy. Subsequently, the excited electron returns to the ground state. During the return course, quantum dots emit light (different color for different quantum dots depending on the size, shape, and material), due to quantum confinement. The presence of target molecules 102 that bound with imprinted sites 810A may consume at least some of the electrons. As a result, light emission from the quantum dots is quenched in the presence of an analyte 104 containing target molecules 102, and the significance of the quenching is proportional to the concentration of the target molecule 102 in analyte 104. The changes in the light intensity and its colour (spectra) are captured by image sensor 828, through the lens 820. The image may be processed by a processor and an image processing software that may analyze one or more spectral characteristics (e.g. the colour (or wavelength) and/or intensity (or irradiance)) to indicate the presence and/or quantity of a target molecule 102. The image analysis may be performed by quantifying the red, green, and blue values of the image signal. The processor may be the processor available in a digital camera or a cellphone. The software may be a software application of a mobile computing device or cloud-based software or the like.

In some embodiments, one or more auxiliary lenses 830, such as a plano-convex lens, or a series of lenses acting as macro lens or wide-angle lens, or super wide-angle lens, may be positioned between detector 828 and sensing layer 812 to shorten the minimum focusing distance of detector 828. This may be advantages for making a sensor 800 with a smaller footprint.

In some embodiments, one or more optical prisms, (not shown) may be positioned between detector 828 and sensing layer 812 to break the output radiation from sensing layer 812 up into its constituent spectral colors. This may be advantages for better detection and processing of the output radiation.

In some embodiments, one or more filters, for example narrowband filters or broadband filters or quantum dots filter (e.g. two-dimensional absorptive filter array composed of colloidal quantum dots) (not shown) may be positioned between the detector 828 and sensing layer 812 to break the output radiation from the sensing layer up into its constituent spectral by means such as measuring light spectrum based on the wavelength multiplexing principle. This may be advantages for better (e.g. more accurate, more favourable detection limit, etc.) detection and processing of the output radiation from sensing layer 812.

In some embodiments, a bandpass filter, a bandstop filter, a UV cut filter, a visible light pass filter, a UV pass filter or visible light cut filter may be employed. In some embodiments, a UV pass filter or visible light cut filter (not shown) to allow passing UV radiation but blocking at least part of the visible light may be applied in front of the radiation source 814, for example. This may be advantages for blocking any visible light radiation emitted from the radiation source (which often inevitably is emitted from solid-state UV sources, such as UV-LEDs) to reach lens 820 and/or detector 828 and interfere with the accuracy of the image capturing and processing (because it may be preferable for only visible light emitted from sensing layer 812 reaches detector 828). In some embodiments, a UV cut filter may be applied in front of detector 828 (not shown) to block at least part of the UV radiation. This may be advantages for blocking any UV radiation emitted from radiation source 814 to reach the lens 820 and/or detector 828 and interfere with the accuracy of the image capturing and processing.

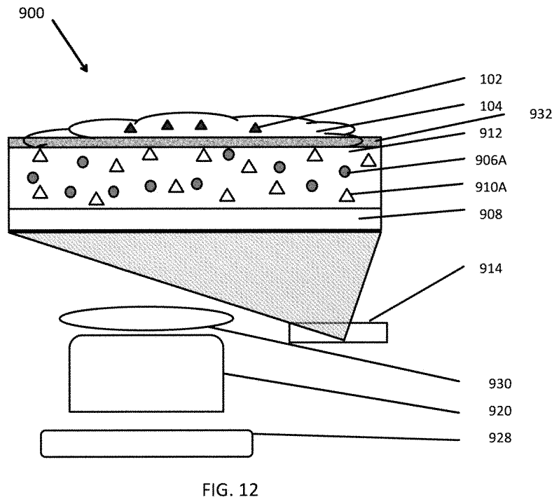

FIG. 12 depicts a sensor device 900 according to an example embodiment of the invention. Sensor device 900 may be substantially similar to sensor 100. Sensor device 900 comprises a radiation source 914 (e.g. substantially similar to radiation source 114) configured to emit input radiation to irradiate a sensing layer 912 immobilized on a radiation transparent substrate 908 (e.g. substantially similar to substrate 108), a detector 928 (e.g. substantially similar to detector 328) and an optional lens 920. Detector 928 may comprise a spectrometer or other types of light detector, and in such case, the lens 920 may be optional or may not be required. Sensing layer 912 comprises sensing material 906A of quantum dots configured to receive radiation from the radiation source 914. Sensing layer 912 also comprises imprinted sites 910A that are configured to selectively interact with (e.g. any form of physical or chemical interactions or binding or reaction, and the like) target molecule(s) 102 or component in analyte 104. When the analyte 104 containing target molecule(s) 102 is brought to contact with sensing layer 912, target molecule(s) 102 may bind to the imprinted sites 910A with high affinity and specificity. In some embodiments, one or more auxiliary lenses 930 may also be present. By locating both radiation source 914 and lens 920 and/or detector 928 on the same side of sensing layer 92, the other side of sensing layer 912 is open (not blocked by detector 928 or radiation source 914) for direct contact with analyte 104. For example, if analyte 104 is the human sweat, sensing layer 912 could be in direct contact with the sweat by different means, for example through being implemented in a wearable device, for example a custom wrist device, a wristband or a watch.

In some embodiments, a perforated or a porous membrane 932 may be positioned on the surface of sensing layer 912 (to be in contact with analyte 104) to block interfering macromolecules. Membrane 932 may be a replicable/disposable membrane that could be changed.

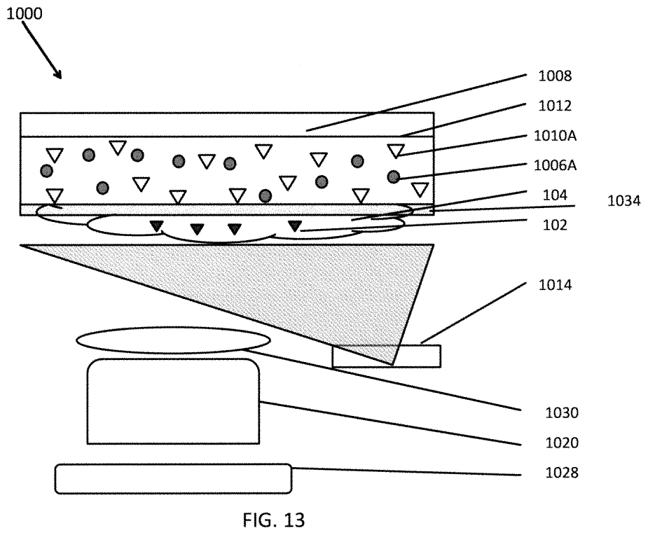

FIG. 13 depicts a sensor device 1000 according to an example embodiment of the invention. Sensor device 1000 may be substantially similar to sensor 100. Sensor device 1000 comprises a radiation source 1014 (e.g. substantially similar to radiation source 114) configured to emit input radiation to irradiate a sensing layer 1012 immobilized on a radiation transparent substrate 1008 (e.g. substantially similar to substrate 108), a detector 1028 (e.g. substantially similar to detector 328) and an optional lens 1020. Detector 1028 may comprise a spectrometer or other types of light detector, and in such case, the lens 1020 may be optional or may not be required. Sensing layer 1012 comprises sensing material 1006A of quantum dots configured to receive radiation from the radiation source 1014. Sensing layer 1012 also comprises imprinted sites 1010A that are configured to selectively interact with (e.g. any form of physical or chemical interactions or binding or reaction, and the like) target molecule(s) 102 or component in analyte 104. When the analyte 104 containing target molecule(s) 102 is brought to contact with sensing layer 1012, target molecule(s) 102 may bind to the imprinted sites 1010A with high affinity and specificity. In some embodiments, one or more auxiliary lenses 1030 may also be present. By locating radiation source 1014 and detector 1028 both on the same side of the sensing layer 1012 that contacts analyte 104, the same side of sensing layer 1012 that is in contact with analyte 104 and target molecules 102 is irradiated and its image is captured by detector 1028, which may provide more accurate recreation of one or more spectral characteristics of the radiation output of sensing layer 1012 (e.g. the colors and readings of the images) and allow for use of non-transparent substrate 1008. For example, sensor 1000 may be applied to the detection of chemical contaminants in water. In such case, if analyte 104 is a water sample, sensing layer 1012 may be put in contact with water by different means, for example through dropping specific volume of water on sensing layer 1012, or by contacting sensing layer 1012 with water for a specific time period. In some embodiments, if analyte 104 is a water sample, for example, sensing layer 1012 may be put in contact with water by applying an absorbent path 1034 on the surface of sensing material 1012, so that a specific quantity of water is in contact with sensing layer 1012. Absorbent path 1034 may be a transparent absorbent path. This may be advantageous as the amount of water in contact with sensing layer 1012 may impact the fluorescent emission from sensing layer 1012. Therefore, for accurate measurement of target molecules 102, it might be desirable to put a specific volume of analyte 104 in contact with sensing layer 1012. In some embodiment test/control lines (lines that change their colors or generate another (image) signal when the water (analyte 104) contact them) may be applied on the opposite side of the side where water was brought in contact with absorbent path 1034, to ensure that water has been absorbed to the entire absorbent path 1034.

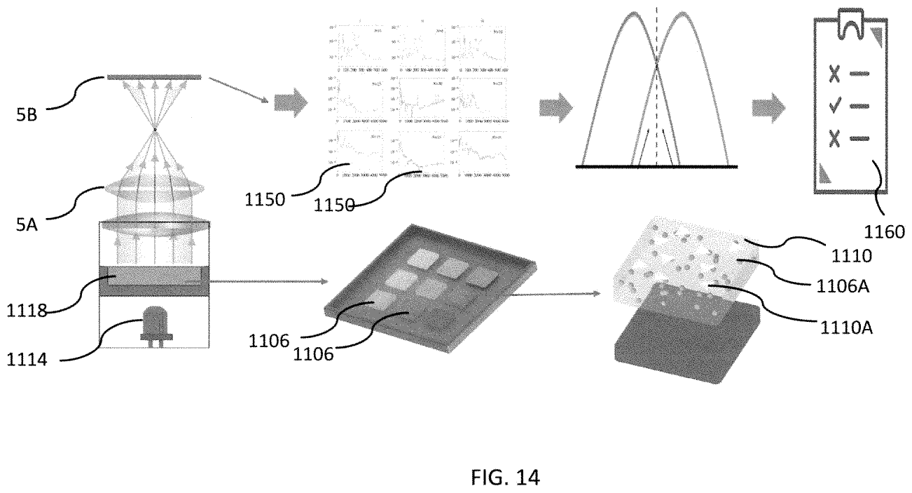

FIG. 14 depicts a sensor device 1100 according to an example embodiment of the invention. Sensor device 1100 may be substantially similar to sensor 200 except in that radiation source 1114 is located behind sensor card 1118 in relation to mobile computing device 5 (of which only lens 5A and detector 5B are shown in FIG. 14). Detector 5B of mobile computing device 5 generates sensing node signals 1150 based on radiation output of sensing nodes 1106 on sensor card 1118. Like other sensing nodes described herein, each sensing node 1106 may comprise recognition element 1110, imprinted sites 1110A, and sensing material 1106A. The signals may be a single value for example the color values of sensing nodes 1106, or could be a curve, for example, changing in the color values of sensing nodes 1106 at different wavelengths or different radiant powers (e.g. irradiances, intensities), where radiation source 1114 may irradiate sensing nodes 1106 at different irradiances or wavelengths during a short period of time. The signals from detector 5B may be analyzed (as discussed herein) to create a report 1160.

### Aspects

The invention includes a number of non-limiting aspects. Non-limiting aspects of the invention comprise:
1. A sensor for detection or quantitative measurement of a first target molecule in an analyte, the sensor comprising:
   a first sensing node provided in solid phase on a solid-phase substrate, the first sensing node comprising a first radiation-activatable fluorescence material and a first recognition element for interaction with the first target molecule;
   a radiation emitter optically configured to direct input radiation toward the first sensing node;
   wherein the first radiation-activatable fluorescence material is fluoresce-able in response to interaction with the input radiation to thereby cause the first sensing node to emit first output radiation; and
   wherein one or more spectral characteristics of the first output radiation are detectably influence-able in response to interaction between the first recognition element and the first target molecule.
2. A sensor according to aspect 1 or any other aspect herein wherein the first recognition element comprises one or more first recognition sites with an affinity for the target molecule.
3. A sensor according to any one of aspects 1 and 2 or any other aspect herein comprising a housing wherein the radiation emitter and the first sensing node are located at least partially within the housing.
4. A sensor according to aspect 3 or any other aspect herein comprising a housing wherein the first sensing node is attached to a wall of the housing.
5. A sensor according to any one of aspects 1 to 4 or any other aspect herein wherein the one or more spectral characteristics of the first output radiation comprise radiation intensity at one or more wavelengths.
6. A sensor according to any one of aspects 1 to 5 or any other aspect herein comprising a detector, wherein the detector is optically configured to capture the one or more spectral characteristics of the first output radiation.
7. A sensor according to aspect 6 or any other aspect herein wherein the detector comprises at least one of: a radiation detector, a light detector, a color detector, an image detector, a digital image sensor, a CCD sensor and a CMOS sensor.
8. A sensor according to any one of aspects 6 and 7 or any other aspect herein comprising at least one of: a narrowband filter, a broadband filter, a bandpass filter, a bandstop filter, a UV pass filter, a UV cut filter, a visible light pass filter, a visible light cut filter and a QDs filter located in at least one of: a first location between the detector and the first sensing node, a second location between the radiation emitter and the detector and a third location between the radiation emitter and the first sensing node.
9. A sensor according to any one of aspects 1 to 8 or any other aspect herein comprising a second sensing node provided in solid phase on the solid-phase substrate, the second sensing node comprising a second radiation-activatable fluorescence material and a second recognition element for interaction with a second target molecule;
   the radiation emitter optically configured to direct the input radiation toward the second sensing node;
   wherein the second radiation-activatable fluorescence material is fluoresce-able in response to interaction with the input radiation to thereby cause the second sensing node to emit second output radiation; and
   wherein one or more spectral characteristics of the second output radiation are detectably influence-able in response to interaction between the second recognition element and the second target molecule. The first recognition element may comprises a first type of recognition site with an affinity for the first target molecule and the second recognition element may comprise a second type of recognition site with an affinity for the second target molecule.
10. A sensor according to any one of aspects 1 to 8 or any other aspect herein comprising a second sensing node provided in solid phase on the solid-phase substrate, the second sensing node comprising a second radiation-activatable fluorescence material;
   the radiation emitter optically configured to direct the input radiation toward the second sensing node;
   wherein the second radiation-activatable fluorescence material is fluoresce-able in response to interaction with the input radiation to thereby cause the second sensing node to emit second output radiation; and
   wherein one or more spectral characteristics of the second output radiation are detectable in response to interaction between the second sensing node and the analyte.
11. A sensor according to any one of aspects 9 and 10 or any other aspect herein wherein the first recognition element is different in shape or chemical composition from the second recognition element.
12. A sensor according to any one of aspects 9 to 11 or any other aspect herein wherein the first recognition element comprises a first type of recognition site with an affinity for the first target molecule and the second recognition element comprises a second type of recognition site with an affinity for the second target molecule.
13. A sensor according to any one of 9 to 12 or any other aspect herein wherein the first target molecule is different in chemical composition from the second target molecule.
14. A sensor according to any one of aspects 1 to 13 or any other aspect herein wherein:
   the first sensing node is provided on a first side of the solid-phase substrate;
   the solid-phase substrate is at least partially transparent to the input radiation emitted by the radiation emitter; and
   the radiation emitter is configured to direct the input radiation toward the first sensing node through the substrate from a second side of the substrate, the second side of the solid-phase substrate different from (e.g. opposite to) the first side of the solid-phase substrate.
15. A sensor according to any one of aspects 1 to 13 or any other aspect herein wherein:
   the solid-phase substrate is provided on a substrate side of the first sensing node; and
   the radiation emitter is configured to direct the input radiation toward a target side of the sensing node, the target side of the sensing node different from (e.g. opposite to) the substrate side of the sensing node.
16. A sensor according to aspect 3 or any other aspect herein wherein at least a portion of the housing is at least partially transparent to the first output radiation such that the one or more spectral characteristics of the first output radiation are detectable through the at least a portion of the housing that is at least partially transparent.
17. A sensor according to aspect 16 or any other aspect herein comprising a detector optically configured to detect the one or more spectral characteristics of the first output radiation through the at least a portion of the housing that is at least partially transparent and wherein the detector comprises at least one of a radiation detector, a light detector, a color detector, an image detector, a digital image sensor, a CCD sensor and a CMOS sensor.
18. A sensor according to aspect 16 or any other aspect herein comprising a detector optically configured to detect the one or more spectral characteristics of the first output radiation through the at least a portion of the housing that is at least partially transparent and wherein the detector comprises a digital image sensor of a mobile computing device.
19. A sensor according to aspect 18 or any other aspect herein wherein the mobile computing device comprises a digital camera, a tablet, a camera phone, a smartphone, a tablet computing device, a smart watch or a smart wearable device.
20. A sensor according to any one of aspects 1 to 19 or any other aspect herein wherein a vector of a principal emission direction of the input radiation is substantially parallel with a vector normal to a plane defined by a broad surface of the first sensing node.
21. A sensor according to any one of aspects 1 to 19 or any other aspect herein wherein a vector of a principal emission direction of input radiation is non-parallel with a vector normal to a plane defined by a broad surface of the first sensing node.
22. A sensor according to any one of aspects 1 to 19 or any other aspect herein wherein a vector of a principal emission direction of the input radiation intersects with a vector normal to a plane defined by a broad surface of the first sensing node by an angle of less than 40 degrees.
23. A sensor according to any one of aspects 1 to 22 or any other aspect herein wherein:
   the radiation emitter is located to irradiate the first sensing node from a substrate side of the sensing node;
   a detector is located on the substrate side of the sensing node to receive first output radiation from the substrate side of the sensing node; and
   the sensing node is located to interact with the analyte on a target side of the sensing node, the target side of the sensing node opposite the substrate side of the sensing node.
24. A sensor according to any one of aspects 1 to 22 or any other aspect herein wherein:
   the radiation emitter is located on to irradiate the first sensing node from a target side of the sensing node;
   a detector is located to receive first output radiation from the target side of the sensing node; and
   the sensing node is located to interact with the analyte on the target side of the sensing node.
25. A sensor according to any one of aspects 1 to 22 or any other aspect herein wherein:
   the radiation emitter is located to irradiate the first sensing node from a substrate side of the sensing node;
   a detector is located to receive first output radiation from a target side of the sensing node; and
   the sensing node is located to interact with the analyte on the target side of the sensing node, the target side of the sensing node opposite the substrate side of the sensing node.
26. A sensor according to any one of aspects 1 to 25 or any other aspect herein comprising an optical lens positioned in an optical path of the first output radiation between the first sensing node and a detector, wherein the detector is configured to measure the one or more spectral characteristics of the first output radiation.
27. A sensor according to any one of aspects 1 to 26 or any other aspect herein comprising a battery to power the radiation emitter and the detector.
28. A sensor according to any one of aspects 1 to 27 or any other aspect herein comprising a repellant module for repelling a first target molecule bound to the first sensing node from the first sensing node, wherein the repellant module comprises at least one of: a pair of electrodes, laser-engraved graphene (LEG), and redox-active nanoreporters (RARs).
29. A sensor according to any one of aspects 1 to 28 or any other aspect herein comprising a release module for stimulating the release of biofluids from skin.
30. A sensor according to aspect 29 or any other aspect herein wherein the release module comprises an iontophoresis module.
31. A sensor according to any one of aspects 1 to 30 or any other aspect herein wherein the first sensing node provided on the solid-phase substrate is removable and replaceable with a second sensing node provided in a solid phase on a second solid-phase substrate, the second sensing node comprising a second radiation-activatable fluorescence material and a second recognition element for interaction with a second target molecule;
   the radiation emitter optically configured to direct the input radiation toward the second sensing node;
   wherein the second radiation-activatable fluorescence material is fluoresce-able in response to interaction with the input radiation to thereby cause the second sensing node to emit second output radiation; and
   wherein one or more spectral characteristics of the second output radiation are detectably influence-able in response to interaction between the second recognition element and the second target molecule.
32. A sensor according to any one of aspects 1 to 31 or any other aspect herein wherein the radiation emitter comprises a solid-state UV emitter.
33. A sensor according to aspect 32 or any other aspect herein wherein the solid-state UV emitter comprises an ultraviolet light emitting diode (UV-LED).
34 A sensor according to any one of aspects 1 to 33 or any other aspect herein wherein the first radiation-activatable fluorescence material comprises one or more types of quantum dots.
35. A sensor according to aspect 34 or any other aspect herein wherein the one or more types of quantum dots comprise at least two types quantum dots and the at least two types of quantum dots comprise types of quantum dots of different chemical compositions, size or shape.
36. A sensor according to any one of aspects 34 and 35 or any other aspect herein wherein the one or more one types of quantum dots comprise at least one type of quantum dot having a chemical composition selected from the group consisting of: zinc oxide (ZnO), cadmium sulfide (CdS), cadmium selenide (CdSe), zinc selenide (ZnSe), indium phosphide (InP), carbon, and graphene.
37. A sensor according to any one of aspects 1 to 36 or any other aspect herein wherein the recognition element comprises an imprinted polymer (IP).
38. A sensor according to aspect 37 or any other aspect herein wherein the imprinted polymer comprises a molecularly imprinted polymer (MIP) or a surface imprinted polymer (SIP).
39. A sensor according to any one of aspects 37 and 38 or any other aspect herein wherein the imprinted polymer comprises at least one of: 3-Aminopropyltriethoxysilane (APTES) or 5-indolyl boronic acid.
40. A sensor according to any one of aspects 1 to 39 or any other aspect herein wherein the first radiation-activatable fluorescence material is doped with at least one of: metal particles, non-metal particles, a catalyst and a polymer.
41. A sensor according to aspect 1 or any other aspect herein comprising at least one of a porous material, microporous material, mesoporous material, macroporous material, ordered hierarchical porous material, structure-directing surfactant, sulfonated tetrafluoroethylene based fluoropolymer-copolymer, crosslinker agent, graphene derivatives, active fluorescent quencher, absorbent path, and membrane integrated with the first sensing node or located between the first sensing node and an analyte-receiving surface of the sensor.
42. A sensor according to any one of aspects 1 to 41 or any other aspect herein wherein the radiation emitter is configurable to emit radiation of at least one of: a plurality of different intensities and a plurality of different wavelengths.
43. A sensor according to any one of aspects 1 to 42 or any other aspect herein wherein the radiation emitter is configurable to emit radiation at different intensities.
44. A sensor according to any one of aspects 1 to 43 or any other aspect herein wherein the radiation emitter comprises a plurality of radiation sub-emitters, wherein at least two sub-emitters are configurable to emit radiation at different wavelengths.
45. A sensor according to any one of aspects 1 to 44 or any other aspect herein comprising:
   a second sensing node provided in solid phase on the solid-phase substrate, the second sensing node comprising a second radiation-activatable fluorescence material and a second recognition element for interaction with a second target molecule;
   a second radiation emitter optically configured to direct second input radiation toward the second sensing node;
   wherein the second radiation-activatable fluorescence material is fluoresce-able in response to interaction with the second input radiation to thereby cause the second sensing node to emit second output radiation;
   wherein one or more spectral characteristics of the second output radiation are detectably influence-able in response to interaction between the second recognition element and the second target molecule; and
   wherein the input radiation and the second input radiation have different intensities and/or different wavelengths.
46. A sensor according to any one of aspects 1 to 45 or any other aspect herein wherein the sensor is integrated into at least one of: a laptop, a mobile phone, a watch, and a wearable device.
47. A sensor according to any one of aspects 1 to 46 or any other aspect herein wherein the first sensing node is fabricated on at least one of: a UV-LED chip, a UV-LED wafer and a UV-LED package.
48. A sensor according to any one of aspects 1 to 46 or any other aspect herein wherein the first sensing node is fabricated on at least one of: paper and polymer sheet substrate.
49. Use of the sensor according to any one of aspects 1 to 48 or any other aspect herein for detecting a presence of, or estimating a quantity of, a target molecule in the analyte.
50. Use according to aspect 49 or any other aspect herein wherein:
   the first target molecule is a biomarker, such as glucose, lactate, dopamine, and/or cortisol, and the analyte is a biofluid, such as sweat, blood, saliva, mucus, urine, stool and/or interstitial fluid.
51. Use according to aspect 49 or any other aspect herein wherein:
   the first target molecule is a pollutant, such as toxic compounds, chemical hazards, or environmental contaminants, and the analyte is air or water.
52. A method for detecting a presence or quantity of a target molecule in an analyte using a sensor, the method comprising:
   establishing contact between the analyte and the first sensing node of the sensor according to any one of aspects 1 to 49 or any other aspect herein; and
   detecting one or more of the one or more spectral characteristics of the first output radiation.
53 A method according to aspect 52 or any other aspect herein wherein the one or more of the one or more spectral characteristics of the first output radiation comprise at least one of: light intensity, light spectrum, light brightness, and a value corresponding to a relative color intensity of at least one of the colors of red, green, blue, cyan, magenta, yellow, and key.
54. A method according to any one of aspects 52 and 53 or any other aspect herein comprising:
   detecting a presence or quantity of the first target molecule in the analyte by employing an artificial intelligence engine trained by machine learning or deep learning to detect the presence or quantity of the target molecule in the analyte based at least in part on the one or more spectral characteristics of the first output radiation.
55. A device for detection or quantitative measurement of a first target molecule in an analyte using the camera of a mobile computing device, the device comprising:
   a radiation emitter supported in a housing and controllable to emit input radiation;
   a solid-phase substrate comprising a first sensing node provided in solid phase on the substrate for exposure to the analyte, the first sensing node comprising a first radiation-activatable fluorescence material and a first recognition element for interaction with the first target molecule in the analyte;
   the solid-phase substrate insertable into the housing in a location where the input radiation impinges on the first sensing node;
   wherein the first radiation-activatable fluorescence material is fluoresce-able in response to interaction with the input radiation to thereby cause the first sensing node to emit output radiation;
   wherein one or more spectral characteristics of the output radiation are detectably influence-able in response to interaction between the first recognition element and the first target molecule;
   wherein the device is mountable, or otherwise locatable, relative to the camera of the mobile computing device such that at least some of the output radiation exits the housing through an aperture and is detectable by the camera.
56. A device according to aspect 55 or any other aspect herein wherein the mobile computing device comprises a digital camera, a tablet, a camera phone, a smartphone, a tablet computing device, a smart watch or a smart wearable device.
57. A device according to any one of aspects 55 and 56 or any other aspect herein wherein the one or more spectral characteristics comprise radiation intensity of the output radiation at one or more wavelengths.
58. A device according to aspect 55 or any other aspect herein comprising any of the features, combinations of features and/or sub-combinations of features of any of aspects 1 to 49.
59. A wearable device for detection or quantitative measurement of a first target molecule in an analyte, the wearable device comprising:
   an image sensor supported in a wearable housing;
   a substrate comprising a first sensing node provided on the substrate, the first sensing node comprising a first radiation-activatable fluorescence material and a first recognition element for interaction with the first target molecule in the analyte;
   the substrate mountable to an exterior of the wearable housing for exposure to the analyte;
   a radiation emitter supported in the wearable housing and controllable to emit input radiation onto the first sensing node;
   wherein the first radiation-activatable fluorescence material is fluoresce-able in response to interaction with the input radiation to thereby cause the sensing node to emit output radiation;
   wherein one or more spectral characteristics of the output radiation are detectably influence-able in response to interaction between the first recognition element and the first target molecule;
   wherein at least some of the output radiation is detectable by the image sensor.
60. A device according to aspect 59 or any other aspect herein wherein the substrate is mountable to an exterior of the housing for exposure to the analyte when the device is being worn.
61. A device according to any one of aspects 59 to 60 or any other aspect herein wherein the image sensor comprises a CMOS image sensor or a CCD image sensor.
62. A device according to any one of aspects 58 to 60 or any other aspect herein wherein the one or more spectral characteristics comprise radiation intensity of the output radiation at one or more wavelengths.
63. A device according to any one of aspects 59 to 62 or any other aspect herein comprising an optical system for at least one of: directing the input radiation onto the first sensing node and directing the output radiation toward the image sensor.
64. A device according to aspect 63 or any other aspect herein wherein the optical system comprises one or more optical elements selected from the group consisting of: mirrors, lenses, fiber optics, prisms, transparent windows and transparent walls.
65. A device according to any one of aspects 59 to 64 or any other aspect herein comprising any of the features, combinations of features and/or sub-combinations of features of any of aspects 1 to 49.

### Interpretation of Terms

Unless the context clearly requires otherwise, throughout the description and the
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. A sensor for detection or quantitative measurement of a first target molecule in an analyte, the sensor comprising:
a first sensing node provided in solid phase on a solid-phase substrate, the first sensing node comprising a first radiation-activatable fluorescence material and a first recognition element for interaction with the first target molecule;
a radiation emitter optically configured to direct input radiation toward the first sensing node;
wherein the first radiation-activatable fluorescence material is fluoresce-able in response to interaction with the input radiation to thereby cause the first sensing node to emit first output radiation; and
wherein one or more spectral characteristics of the first output radiation are detectably influence-able in response to interaction between the first recognition element and the first target molecule.

2. A sensor according to claim 1 or any other claim herein wherein the first recognition element comprises one or more first recognition sites with an affinity for the target molecule.

3. A sensor according to claim 1 comprising a housing wherein the radiation emitter and the first sensing node are located at least partially within the housing.

4. A sensor according to claim 3 comprising a housing wherein the first sensing node is attached to a wall of the housing.

5. A sensor according to claim 1 wherein the one or more spectral characteristics of the first output radiation comprise radiation intensity at one or more wavelengths.

6. A sensor according to claim 1 comprising a detector, wherein the detector is optically configured to capture the one or more spectral characteristics of the first output radiation.

7. A sensor according to claim 6 wherein the detector comprises at least one of: a radiation detector, a light detector, a color detector, an image detector, a digital image sensor, a CCD sensor and a CMOS sensor.

8. A sensor according to claim 6 comprising at least one of: a narrowband filter, a broadband filter, a bandpass filter, a bandstop filter, a UV pass filter, a UV cut filter, a visible light pass filter, a visible light cut filter and a QDs filter located in at least one of: a first location between the detector and the first sensing node, a second location between the radiation emitter and the detector and a third location between the radiation emitter and the first sensing node.

9. A sensor according to claim 1 comprising a second sensing node provided in solid phase on the solid-phase substrate, the second sensing node comprising a second radiation-activatable fluorescence material and a second recognition element for interaction with a second target molecule;
the radiation emitter optically configured to direct the input radiation toward the second sensing node;
wherein the second radiation-activatable fluorescence material is fluoresce-able in response to interaction with the input radiation to thereby cause the second sensing node to emit second output radiation; and
wherein one or more spectral characteristics of the second output radiation are detectably influence-able in response to interaction between the second recognition element and the second target molecule.

10. A sensor according to claim 1 comprising a second sensing node provided in solid phase on the solid-phase substrate, the second sensing node comprising a second radiation-activatable fluorescence material;
the radiation emitter optically configured to direct the input radiation toward the second sensing node;
wherein the second radiation-activatable fluorescence material is fluoresce-able in response to interaction with the input radiation to thereby cause the second sensing node to emit second output radiation; and
wherein one or more spectral characteristics of the second output radiation are detectable in response to interaction between the second sensing node and the analyte.

11. A sensor according to claim 1 wherein:
the first sensing node is provided on a first side of the solid-phase substrate;
the solid-phase substrate is at least partially transparent to the input radiation emitted by the radiation emitter; and
the radiation emitter is configured to direct the input radiation toward the first sensing node through the substrate from a second side of the substrate, the second side of the solid-phase substrate different from (e.g. opposite to) the first side of the solid-phase substrate.

12. A sensor according to claim 3 wherein at least a portion of the housing is at least partially transparent to the first output radiation such that the one or more spectral characteristics of the first output radiation are detectable through the at least a portion of the housing that is at least partially transparent.

13. A sensor according to claim 12 comprising a detector optically configured to detect the one or more spectral characteristics of the first output radiation through the at least a portion of the housing that is at least partially transparent and wherein the detector comprises at least one of a radiation detector, a light detector, a color detector, an image detector, a digital image sensor, a CCD sensor and a CMOS sensor.

14. A sensor according to claim 12 comprising a detector optically configured to detect the one or more spectral characteristics of the first output radiation through the at least a portion of the housing that is at least partially transparent and wherein the detector comprises a digital image sensor of a mobile computing device.

15. A sensor according to claim 14 wherein the mobile computing device comprises a digital camera, a tablet, a camera phone, a smartphone, a tablet computing device, a smart watch or a smart wearable device.

16. A sensor according to claim 1 wherein:
the radiation emitter is located to irradiate the first sensing node from a substrate side of the sensing node;
a detector is located on the substrate side of the sensing node to receive first output radiation from the substrate side of the sensing node; and
the sensing node is located to interact with the analyte on a target side of the sensing node, the target side of the sensing node opposite the substrate side of the sensing node.

17. A sensor according to claim 1 comprising an optical lens positioned in an optical path of the first output radiation between the first sensing node and a detector, wherein the detector is configured to measure the one or more spectral characteristics of the first output radiation.

18. A sensor according to claim 1 comprising a repellant module for repelling a first target molecule bound to the first sensing node from the first sensing node, wherein the repellant module comprises at least one of: a pair of electrodes, laser-engraved graphene (LEG), and redox-active nanoreporters (RARs).

19. A sensor according to claim 1 comprising a release module for stimulating the release of biofluids from skin.

20. A sensor according to claim 1 wherein the first sensing node provided on the solid-phase substrate is removable and replaceable with a second sensing node provided in a solid phase on a second solid-phase substrate, the second sensing node comprising a second radiation-activatable fluorescence material and a second recognition element for interaction with a second target molecule;
the radiation emitter optically configured to direct the input radiation toward the second sensing node;
wherein the second radiation-activatable fluorescence material is fluoresce-able in response to interaction with the input radiation to thereby cause the second sensing node to emit second output radiation; and
wherein one or more spectral characteristics of the second output radiation are detectably influence-able in response to interaction between the second recognition element and the second target molecule.

21. A sensor according to claim 1 wherein the radiation emitter comprises a solid-state UV emitter.

22. A sensor according to claim 1 wherein the first radiation-activatable fluorescence material comprises one or more types of quantum dots.

23. A sensor according to claim 1 wherein the recognition element comprises an imprinted polymer (IP).

24. A sensor according to claim 1 comprising at least one of a porous material, microporous material, mesoporous material, macroporous material, ordered hierarchical porous material, structure-directing surfactant, sulfonated tetrafluoroethylene based fluoropolymer-copolymer, crosslinker agent, graphene derivatives, active fluorescent quencher, absorbent path, and membrane integrated with the first sensing node or located between the first sensing node and an analyte-receiving surface of the sensor.

25. A sensor according to claim 1 wherein the radiation emitter is configurable to emit radiation of at least one of: a plurality of different intensities and a plurality of different wavelengths.

26. A sensor according to claim 1 wherein the sensor is integrated into at least one of: a laptop, a mobile phone, a watch, and a wearable device.

27. A sensor according to claim 1 wherein the first sensing node is fabricated on at least one of: a UV-LED chip, a UV-LED wafer and a UV-LED package.

28. A sensor according to claim 1 wherein the first sensing node is fabricated on at least one of: paper and polymer sheet substrate.

29. A method for detecting a presence or quantity of a target molecule in an analyte using a sensor, the method comprising:
establishing contact between the analyte and the first sensing node of the sensor according to claim 1; and
detecting one or more of the one or more spectral characteristics of the first output radiation.

30. A method according to claim 29 wherein the one or more of the one or more spectral characteristics of the first output radiation comprise at least one of: light intensity, light spectrum, light brightness, and a value corresponding to a relative color intensity of at least one of the colors of red, green, blue, cyan, magenta, yellow, and key.

31. A method according to claim 29 comprising:
detecting a presence or quantity of the first target molecule in the analyte by employing an artificial intelligence engine trained by machine learning or deep learning to detect the presence or quantity of the target molecule in the analyte based at least in part on the one or more spectral characteristics of the first output radiation.

32. A device for detection or quantitative measurement of a first target molecule in an analyte using the camera of a mobile computing device, the device comprising:
a radiation emitter supported in a housing and controllable to emit input radiation;
a solid-phase substrate comprising a first sensing node provided in solid phase on the substrate for exposure to the analyte, the first sensing node comprising a first radiation-activatable fluorescence material and a first recognition element for interaction with the first target molecule in the analyte;
the solid-phase substrate insertable into the housing in a location where the input radiation impinges on the first sensing node;
wherein the first radiation-activatable fluorescence material is fluoresce-able in response to interaction with the input radiation to thereby cause the first sensing node to emit output radiation;
wherein one or more spectral characteristics of the output radiation are detectably influence-able in response to interaction between the first recognition element and the first target molecule;
wherein the device is mountable, or otherwise locatable, relative to the camera of the mobile computing device such that at least some of the output radiation exits the housing through an aperture and is detectable by the camera.

33. A wearable device for detection or quantitative measurement of a first target molecule in an analyte, the wearable device comprising:
an image sensor supported in a wearable housing;
a substrate comprising a first sensing node provided on the substrate, the first sensing node comprising a first radiation-activatable fluorescence material and a first recognition element for interaction with the first target molecule in the analyte;
the substrate mountable to an exterior of the wearable housing for exposure to the analyte;
a radiation emitter supported in the wearable housing and controllable to emit input radiation onto the first sensing node;
wherein the first radiation-activatable fluorescence material is fluoresce-able in response to interaction with the input radiation to thereby cause the sensing node to emit output radiation;
wherein one or more spectral characteristics of the output radiation are detectably influence-able in response to interaction between the first recognition element and the first target molecule;
wherein at least some of the output radiation is detectable by the image sensor.

34. A device according to claim 33 wherein the substrate is mountable to an exterior of the housing for exposure to the analyte when the device is being worn.

35. A device according to claim 33 comprising an optical system for at least one of: directing the input radiation onto the first sensing node and directing the output radiation toward the image sensor.
